(19) United States
(12) Patent Application Publication
(10) Pub. No.: US 2019/0275138 A1
(43) Pub. Date: Sep. 12, 2019

(54) FOOT AND MOUTH DISEASE VIRUS SEROTYPE O (FMDV-O) VACCINE

(71) Applicant: The Wistar Institute of Anatomy and Biology, Philadalelphia, PA (US)

(72) Inventors: David B. Weiner, Merion, PA (US); Kar Muthumani, Cherry Hill, NJ (US)

(21) Appl. No.: 16/462,649

(22) PCT Filed: Nov. 22, 2017
(86) PCT No.: PCT/US2017/062936

§ 371 Date: May 21, 2019

Related U.S. Application Data
(60) Provisional application No. 62/425,388, filed on Nov. 22, 2016.

(51) Int. Cl. A61K 39/12; C07K 14/005

(52) U.S. Cl. CPC A61K 39/12; C07K 14/005; A61K 2039/53; C12N 2770/32134; C12N 2770/32171; C12N 2770/32122

(57) ABSTRACT

The present invention relates to synthetic, consensus foot-and-mouth disease virus serotype O (FMDV-O) immunogenic proteins and nucleic acid molecule encoding such proteins and to methods for inducing immune responses against FMVD-O.

### CROSS REFERENCE TO RELATED APPLICATIONS

This application is entitled to priority to U.S. Provisional Application No. 62/425,388, filed Nov. 22, 2016, which is incorporated by reference herein in its entirety.

### FIELD OF THE INVENTION

The present invention relates to synthetic, consensus foot-and-mouth disease virus serotype O (FMDV-O) immunogenic proteins and nucleic acid molecule encoding such proteins, vaccines against FMDV-O, methods for inducing immune responses against FMVD-O, and methods of prophylactically and/or therapeutically immunizing individuals against FMDV-O.

### BACKGROUND OF THE INVENTION

Foot-and-mouth disease (FMD) is a highly contagious disease of domestic and wild cloven-hoofed animals including cattle, swine, goats and deer which rapidly replicates in the host and spreads to in-contact susceptible animals. The disease is characterized by fever, lameness, and vesicular lesions of the tongue, feet, snout, and teats resulting in high morbidity, but low mortality in adult animals. FMDV infection drives an acute vesicular disease in cattle, buffaloes, sheep, goats and pigs, which may develop into persistent infection (excluding pigs). FMDV can infect many other mammalian species, including antelopes, elephants, hedgehogs, among others. It is though that the original FMDV natural host might be the African buffalo since: i) it is persistently infected and ii) disease is rarely observed.

The causative agent of FMD is the foot-and-mouth disease virus (FMDV), a Group IV (+) ssRNA virus of the Aphthovirus genus, of the Picornaviridae family. FMDV occurs in seven major serotypes: O, A, C, SAT-1, SAT-2, SAT-3, and Asia-1. These serotypes are regionally restricted with the 0 serotype most common world-wide. The single-stranded, positive-sense RNA genome of FMDV is approximately 8500 bases surrounded by an icosahedral capsid with 60 copies each of four structural proteins VP1-VP4. The viral proteins are antigenically highly variable within its several serotypes including A, Asia 1, O. C, SAT1, SAT2, and SAT3.

FMD is economically devastating and infection of cloven-hoofed livestock can result in significant losses. Recent outbreaks have resulted in billions of dollars lost. Outbreaks have recently occurred in a number of previously disease free countries including Taiwan in 1997, United Kingdom and Netherlands in 2001, and the emergence in several South American countries has risen the awareness of the economically destructive virus. Furthermore, there is world-wide concern that a possible economic terrorist attack employing FMDV to target countries with large livestock industries, such as the US $100 billion/year livestock industry.

Previous measures to control FMDV include slaughter of the infected or in-contact animals and decontamination. Countries that slaughter their livestock due to a FMDV outbreak can only resume livestock activities if the countries have FMDV free status for 3 months after the last outbreak. Countries usually use vaccination of the animals to treat an FMDV outbreak as a last resort because countries that have vaccinated and do not slaughter the animals must wait an entire year to regain FMD free status. Countries, however, are looking to vaccinate their animals before any FMDV outbreak and would be able to retain their FMD free status.

In the past, FMDV vaccines included chemically inactivated whole virus antigen in conjunction with an adjuvant; however, there are disadvantages to this because it requires expensive high-containment manufacturing facilities to produce the vaccine. Over the past 25-30 years investigators have been trying to develop a vaccine that provides protection after a single inoculation.

Accordingly, there is a need in the art for a vaccine that is suitable to provide protection against FDMV-O.

### SUMMARY OF THE INVENTION

The invention relates to a nucleic acid molecule comprising sequences encoding a synthetic consensus FMDV-O viral protein. In one embodiment the nucleic acid molecule comprises VP4 linked at its C terminus to a protease cleavage site linked at its C terminus to viral protein VP2 linked at its C terminus to a protease cleavage site linked at its C terminus to viral protein VP3 linked at its C terminus to a protease cleavage site linked to viral protein VP1 at its C terminus to a protease cleavage site linked to viral protein 2A. The nucleic acid molecule may further comprise a nucleic acid sequence that encodes a leader sequence at the 5′ end of coding sequence for viral protein VP4. In some embodiments, the coding sequence for viral protein VP4 is omitted. In some embodiments, the coding sequence for viral protein 2A is omitted. In some embodiments, the coding sequence that encodes the N terminal leader sequence is omitted. In some embodiments, the coding sequence that encodes the N terminal leader sequence is an Ig leader sequence such as an IgG or IgE leader sequence. In some embodiments the cleavage site is recognized by furin. Plasmids comprising the nucleic acid molecules are also provided.

In one embodiment, the nucleic acid molecule encodes at least one synthetic consensus FMDV-O VP antigen. In one embodiment, the nucleic acid molecule encodes a synthetic consensus FMDV-O VP1 antigen.

Nucleic acid molecule comprising sequences encoding viral protein VP4 linked at its C terminus to sequences encoding a protease cleavage site linked at its C terminus to sequences encoding viral protein VP2 linked at its C terminus to sequences encoding a protease cleavage site linked at its C terminus to sequences encoding viral protein VP3 linked at its C terminus to sequences encoding a protease cleavage site linked to sequences encoding viral protein VP1 linked at its C terminus to sequences encoding viral protein 2A are disclosed herein are referred to long versions or “long”. Nucleic acid molecule comprising sequences encoding viral protein VP2 linked at its C terminus to sequences encoding a protease cleavage site linked at its C terminus to sequences encoding viral protein VP3 linked at its C terminus to sequences encoding a protease cleavage site linked to sequences encoding viral protein VP1 linked at its C terminus to sequences encoding viral protein 2A are disclosed herein are referred to short versions or “short”. In both long and short versions, coding sequence for the protease cleavage site that linked to the 3′ end of the coding sequence encoding viral protein VP1 and coding sequence for viral protein 2A linked may be omitted. Coding sequence for an N terminal leader sequence may be operably linked to the N terminal of coding sequence for an FMDV-O VP. The N terminal leader is preferable an Ig leader such as an IgG or an IgE signal sequence. In some embodiments the cleavage site is recognized by furin.

Methods of generating an immune response against FMDV-O in a subject by administering to the subject one of the disclosed immunogenic compositions are provided.

Methods of preventing infection by FMDV-O in a subject by administering to the subject one of the disclosed immunogenic compositions are provided.

Provided herein is an isolated nucleic acid comprising a sequence encoding the consensus amino acid sequence of at least one VP, and preferably, VP1-VP4 of foot-and-mouth disease virus serotype O that elicits an immune response in a subject. The nucleic acid may comprise a sequence selected from the group consisting of (a) a construct derived from FMDV-O encoding VP4-VP2-VP3-VP1 as set forth in SEQ ID NO:12 and (b) a construct derived from FMDV-O encoding VP4-VP2-VP3-VP1 operably linked to an IgE leader sequence as set forth in SEQ ID NO:14.

In one embodiment, a construct derived from FMDV-O encoding VP4-VP2-VP3-VP1 as set forth in SEQ ID NO:12 comprises a nucleotide sequence as set forth in SEQ ID NO:11. In one embodiment, a construct derived from FMDV-O encoding VP4-VP2-VP3-VP1 operably linked to an IgE leader sequence as set forth in SEQ ID NO:14 comprises a nucleotide sequence as set forth in SEQ ID NO:13.

Nucleic acid molecules in compositions may comprise the following nucleic acid sequences, and/or fragments thereof, and/or homologous sequences to the sequences, and/or fragments of such homologous sequences; the nucleic acid sequence being: (a) a nucleotide sequence encoding FMDV-O derived VP4-VP2-VP3-VP1 as set forth in SEQ ID NO:12 and (b) a nucleotide sequence encoding FMDV-O derived VP4-VP2-VP3-VP1 operably linked to an IgE leader sequence as set forth in SEQ ID NO:14. The amino acid sequence of the cleavage site recognized by the protease furin is set forth in SEQ ID NO:15.

Also provided herein is an immunogenic composition capable of generating in a mammal an immune response against FMDV-O where the immunogenic composition comprises a DNA plasmid comprising a promoter operably linked to a coding sequence that encoding consensus FMDV-O VP1-VP4 antigens and a pharmaceutically acceptable excipient wherein the DNA plasmid is capable of expressing the consensus FMDV-O antigens in a cell of the mammal in a quantity effective to elicit an immune response in the mammal.

The immunogenic composition may be administered to a mammal such as swine, ruminant, human or a primate. The immunogenic composition may elicit an immune response in a mammal such as a humoral, cellular, or both a humoral and cellular response.

Also provided herein is an immunogenic composition capable of generating in a mammal an immune response against a FDMV-O where the immunogenic composition comprises one or more consensus VP antigen of FDMV-O and a pharmaceutically acceptable excipient. The pharmaceutically acceptable excipient may be an adjuvant selected from the group consisting of IL-2 and IL-15. The pharmaceutically acceptable excipient of the immunogenic composition may be transfection facilitating agent. The transfection facilitating agent may be a polyanion, polycation or a lipid such as poly-L-glutamate at a concentration of less than 6mg/ml. The immunogenic composition may be administered to a mammal such as a swine, ruminant, human or primate. The immunogenic composition may elicit an immune response in a mammal such as a humoral, cellular, or both a humoral and cellular response.

Also provided herein is a method for eliciting an immune response against FMDV-O in a mammal comprising delivering the immunogenic composition comprising a DNA plasmid described herein to the tissue of the mammal and electroporating cells of the tissue with a pulse of energy at a constant current effective to permit entry of the DNA plasmid into the cells. The delivery of the DNA plasmids described herein may be accomplished by a method may comprise injecting the DNA plasmid into the intradermic, subcutaneous, or muscle tissue. The DNA plasmid of the method may be delivered by presetting the current and the pulse of energy is at a constant current that equals the present current. The electroporation step of the method may further comprise measuring the impedance in the electroporated cells, adjusting the energy level of the pulse of energy relative to the measured impedance to maintain a constant current in the electroporated cells wherein the measuring and adjusting step occurs within a lifetime of the pulse of energy. The electroporating step may further comprise delivering the pulse of energy to a plurality of electrodes according to a pulse sequence pattern that delivers the pulse of energy in a decentralized pattern.

### BRIEF DESCRIPTION OF DRAWINGS

FIG. 1 shows a phylogenetic analysis of FMDV-O and the VP1 consensus construct. The star indicates the synthetic consensus construct, relative to divergent FMDV-O viruses.

FIG. 2 shows schematic representation of a FMDV-O DNA vaccine construct. The consensus FMDV-O construct comprises VP4, VP2, VP3, VP1 and 2A with cleavage sites in between, operably linked to an IgE leader sequence. The FMDV-O construct is cloned into BamH1 and Xho-1 sites of an expression plasmid to form pFMDV-O.

FIG. 3 shows an image of a gel electrophoresis analysis demonstrating the consensus FMDV-O construct was inserted into the expression plasmid. The sequence included an IgE leader sequence at the N terminus (shaded) and proteolytic cleavage sites (underlined). The VP4 sequences is shown between the IgE leader and first proteolytic cleavage site. The VP2 sequences is shown between the first and second proteolytic cleavage site. The VP3 sequences is shown between the second and third proteolytic cleavage site. The VP1 sequence shown between the third and fourth proteolytic cleavage site and a 2a sequence between the last (fourth) proteolytic cleavage site and stop.

FIG. 4 shows experimental results of an immunofluorescent assay showing staining of 293T cells transfected with pFMDV-O.

FIG. 5 shows an experimental protocol of immunization experiments using electroporation to evaluate immune responses following administration of 1) control plasmid or 2) pFMDV-O.

FIG. 6 shows data of cellular immune responses elicited by FMDV-O vaccination. FMDV-O-VP1s immunization scheme induced significant differences in cell-mediated immune responses and strongly react with FMDV-O peptides. Groups of mice (n=4/group) were immunized three times, each 2 weeks apart with 25 ug FMDV-O DNA vaccine. Samples were collected a week after the third immunization (day 35). FMDV-O-specific CD8 T-lymphocyte responses were assessed by IFN-γ ELISpot assays to peptide pools of overlapping FMDV-VP1 peptides. Mean responses were measured in each pool in all groups one week after the third immunization.

FIG. 7, comprising FIG. 7A and FIG. 7B, shows data of antibody induction in mice elicited by FMDV-0 vaccine. FIG. 7A depicts aystemic anti-FMDV IgG levels after DNA immunization. Each group of Balb/C mice (n=4) was immunized with FMDV-O vaccine construct. Mice were bled 1 week after third immunization, and sera (n=4) was diluted as indicted. ELISA plates were coated with VP-1 protein. Each data point is the average absorbance from triplicate wells. FIG. 7B depicts vaccination and electroporation of mice with FMDV-O VP1s immunogens elicits a considerable antibody response which reacts with FMDV antigen.

### DETAILED DESCRIPTION

Consensus amino acid sequences have been generated for fusion proteins comprising multiple FMDV VPs from FMDV serotype O. Nucleic acid molecules encoding the proteins have also been generated.

In one aspect of the present invention, there are fusion proteins comprising FMDV-O proteins VP1, VP2, VP3, VP4 and/or 2A, and nucleic acid sequences encoding these proteins, which can be generated and used in an immunogenic composition to provide protection of mammals against FMDV-O.

Constructs of some embodiments provide viral proteins VP1, VP2, VP3, and VP4 in a specific order: VP4 -VP2 -VP3 -VP1. An optional tail, 2A is also provided. The constructs have an optional IgE leader sequence. When expressed, a proteolytic cleavage site “CS” is provided between each of VP4, VP2, VP3, VP1 and when present 2A. The protease which can process the site may be furin in some embodiments or a FMDV protease in some embodiments. Other protease sites may be used. The site must be recognized by a protease commonly found in cells where the immunogenic composition is expressed.

In one aspect of the present invention, there are fusion proteins comprising consensus FMDV-O proteins VP1, VP2, VP3, VP4 and/or 2A and nucleic acid sequences encoding these proteins, which can be generated and used in an immunogenic composition to provide protection of mammals against FMDV-O.
1. Definitions.

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

a. Adjuvant

“Adjuvant” as used herein may mean any molecule added to the DNA plasmid based immunogenic compositions or vaccines described herein to enhance antigenicity of the foot-and-mouth disease virus (FMDV) antigen encoded by the DNA plasmids and encoding nucleic acid sequences described hereinafter.

b. Antibody

“Antibody” may mean an antibody of classes IgG, IgM, IgA, IgD or IgE, or fragments, fragments or derivatives thereof, including Fab, F(ab′)2, Fd, and single chain antibodies, diabodies, bispecific antibodies, bifunctional antibodies and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

c. Coding Sequence

“Coding sequence” or “encoding nucleic acid” as used herein may mean refers to the nucleic acid (RNA or DNA molecule) that comprise a nucleotide sequence which encodes a protein. The coding sequence may further include initiation and termination signals operably linked to regulatory elements including a promoter and polyadenylation signal capable of directing expression in the cells of an individual or mammal to which the nucleic acid is administered.

d. Complement

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

e. Consensus or Consensus Sequence

“Consensus” or “consensus sequence” as used herein may mean a synthetic nucleic acid sequence, or corresponding polypeptide sequence, constructed based on analysis of an alignment of multiple sequences of a particular influenza antigen, that can be used to induce broad immunity against the influenza antigen. Consensus FMDV antigens may include VP1, VP2, VP3, VP4, and 2A peptide nucleotide and amino acid sequences.

f. Constant Current

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

g. Current Feedback or Feedback

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

h. Decentralized Current

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

i. Electroporation

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

j. Feedback Mechanism

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

k. Fragment

“Fragment” as used herein may refer a percentage of a full length polypeptide or nucleic acid sequence. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the full length of the parental full length polypeptide or nucleic acid sequence or variant thereof.

l. Homology

“Homology” for nucleotides and amino acids as used herein may be determined using FASTA, BLAST and Gapped BLAST (Altschul et al., Nuc. Acids Res., 1997, 25, 3389, which is incorporated herein by reference in its entirety) and PAUP* 4.0b10 software (D. L. Swofford, Sinauer Associates, Massachusetts). Briefly, the BLAST algorithm, which stands for Basic Local Alignment Search Tool is suitable for determining sequence similarity (Altschul et al., J. Mol. Biol., 1990, 215, 403-410, which is incorporated herein by reference in its entirety). Software for performing BLAST analyses is publicly available through the National Center for Biotechnology Information.

m. Identical

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

n. Impedance

“Impedance” as used herein may be used when discussing the feedback mechanism and can be converted to a current value according to Ohm's law, thus enabling comparisons with the preset current.

o. Immune Response

“Immune response” as used herein may mean the activation of a host's immune system, e.g., that of a mammal, in response to the introduction of FMDV consensus antigen via the provided DNA plasmid based immunogenic compositions or vaccines. The immune response can be in the form of a cellular or humoral response, or both.

p. Nucleic Acid

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH2, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference. Modified nucleotides and nucleic acids may also include locked nucleic acids (LNA), as described in U.S. Patent No. 20020115080, which is incorporated herein by reference. Additional modified nucleotides and nucleic acids are described in U.S. Patent Publication No. 20050182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

q. Operably Linked

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

r. Promoter

“Promoter” as used herein may mean a synthetic or naturally-derived molecule which is capable of conferring, activating or enhancing expression of a nucleic acid in a cell.

A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

s. Stringent Hybridization Conditions

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium).

Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

t. Substantially Complementary

“Substantially complementary” as used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

u. Substantially Identical

“Substantially identical” as used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

v. Subtype or Serotype

“Subtype” or “serotype” as used herein interchangeably and means genetic variants of a virus or viral antigen such that one serotype is recognized by an immune system apart from a different serotype.

w. Variant

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change.

These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

x. Vector

“Vector” used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.
2. Description

The invention provides an optimized consensus sequence encoding a FMDV-O antigen. In one embodiment, the FMDV-O antigen encoded by the optimized consensus sequence is capable of eliciting an immune response in a mammal. In one embodiment, the FMDV-O antigen encoded by the optimized consensus sequence can comprise an epitope(s) that makes it particularly effective as an immunogen against which an immune response can be induced.

The optimized consensus sequence can be a consensus sequence derived from two or more FMDV-O proteins. The optimized consensus sequence can comprise a consensus sequence and/or modification(s) for improved expression. Modification can include codon optimization, RNA optimization, addition of a kozak sequence for increased translation initiation, and/or the addition of an immunoglobulin leader sequence to increase immunogenicity. The FMDV-O antigen encoded by the optimized consensus sequence can comprise a signal peptide such as an immunoglobulin signal peptide, for example, but not limited to, an immunoglobulin E (IgE) or immunoglobulin (IgG) signal peptide. In some embodiments, the antigen encoded by the optimized consensus sequence can comprise a hemagglutinin (HA) tag. The antigen encoded by the optimized consensus sequence can be designed to elicit stronger cellular and/or humoral immune responses than a corresponding non-optimized antigen.

Provided herein are FMDV-O immunogens that can be used to induce broad immunity against multiple subtypes or serotypes of FMDV. In one embodiment, the present invention provides an immunogenic composition comprising one or more nucleic acid molecules that are capable of generating in a mammal an immune response against FMDV. The present invention also provides isolated nucleic acid molecules that are capable of generating in a mammal an immune response against FMDV. In one embodiment, the nucleic acid molecule comprises an optimized nucleotide sequence encoding a consensus FMDV-O immunogen.

In one embodiment, the invention provides compositions comprising a consensus FMDV-O viral capsid protein (VP) immunogens, or fragments or variants thereof, or nucleic acid molecules encoding the same. FMDV-O VP immunogens include VP1, VP2, VP3 and VP4.

Amino acid sequences for consensus FMDV-O VP immunogens are set forth in SEQ ID NO:2, SEQ ID NO:4, SEQ ID NO:6 and SEQ ID NO:8. Therefore, in various embodiments, the invention relates to compositions comprising SEQ ID NO:2, SEQ ID NO:4, SEQ ID NO:6 and SEQ ID NO:8, variants thereof, fragments of SEQ ID NO:2, SEQ ID NO:4, SEQ ID NO:6, SEQ ID NO:8, and variants thereof, and nucleic acid molecules encoding the same.

In one embodiment, the invention provides compositions comprising a nucleic acid molecule comprising a nucleotide sequence that encodes a consensus FMDV-O VP immunogen. Nucleotide sequences of the invention may encode one or more of a consensus FMDV-O VP1 immunogen, a consensus FMDV-O VP2 immunogen, a consensus FMDV-O VP3 immunogen, and a consensus FMDV-O VP4 immunogen.

In one embodiment, a nucleotide sequence which encodes a consensus FMDV-O VP1 immunogen encodes SEQ ID NO:2. In one embodiment, a nucleotide sequence which encodes a consensus FMDV-O VP1 immunogen is SEQ ID NO:l.

In one embodiment, a nucleotide sequence which encodes a consensus FMDV-O VP2 immunogen encodes SEQ ID NO:4. In one embodiment, a nucleotide sequence which encodes a consensus FMDV-O VP2 immunogen is SEQ ID NO:3.

In one embodiment, a nucleotide sequence which encodes a consensus FMDV-O VP3 immunogen encodes SEQ ID NO:6. In one embodiment, a nucleotide sequence which encodes a consensus FMDV-O VP3 immunogen is SEQ ID NO:5.

In one embodiment, a nucleotide sequence which encodes a consensus FMDV-O VP4 immunogen encodes SEQ ID NO:8. In one embodiment, a nucleotide sequence which encodes a consensus FMDV-O VP4 immunogen is SEQ ID NO:7.

In one embodiment, the invention provides compositions comprising a nucleic acid molecule comprising a nucleotide sequence that encodes multiple consensus FMDV-O VP immunogens. Nucleotide sequences of the invention may encode 2, 3 or each of a consensus FMDV-O VP1 immunogen, a consensus FMDV-O VP2 immunogen, a consensus FMDV-O VP3 immunogen, and a consensus FMDV-O VP4 immunogen.

In one embodiment, a nucleotide sequence which encodes each of a consensus FMDV-O VP1 immunogen, a consensus FMDV-O VP2 immunogen, a consensus FMDV-O VP3 immunogen, and a consensus FMDV-O VP4 immunogen encodes SEQ ID NO:12 or SEQ ID NO:14. In one embodiment, a nucleotide sequence which encodes each of a consensus FMDV-O VP1 immunogen, a consensus FMDV-O VP2 immunogen, a consensus FMDV-O VP3 immunogen, and a consensus FMDV-O VP4 immunogen is SEQ ID NO:11 or SEQ ID NO:13.

In one embodiment, at least one optimized consensus encoded FMDV-O antigen is operably linked to one or more regulatory elements. In one embodiment, a regulatory element is a leader sequence. In one embodiment, the leader sequence is an IgE leader sequence. In one embodiment, the IgE leader sequence has an amino acid sequence as set forth in SEQ ID NO:16. Therefore in one embodiment, the invention relates to an amino acid sequence as set forth in SEQ ID NO:2, SEQ ID NO:4, SEQ ID NO:6, SEQ ID NO:8 or SEQ ID NO:12 operably linked to an amino acid sequence as set forth in SEQ ID NO:16. In one embodiment, the invention relates to a nucleotide sequence encoding an amino acid sequence as set forth in SEQ ID NO:2, SEQ ID NO:4, SEQ ID NO:6, SEQ ID NO:8 or SEQ ID NO:12 operably linked to a nucleotide sequence encoding SEQ ID NO:16.

In one embodiment, a regulatory element is a start codon. Therefore, in one embodiment, the invention relates to a nucleic acid sequence as set forth in SEQ ID NO:1, SEQ ID NO:3, SEQ ID NO:5 or SEQ ID NO:7, or a fragment or homolog thereof, operably linked to a nucleotide sequence comprising a start codon at the 5′ terminus. In one embodiment, the invention relates to an amino acid sequence as set forth in SEQ ID NO:2, SEQ ID NO:4, SEQ ID NO:6, or SEQ ID NO:8 or a fragment or homolog thereof, operably linked to an amino acid encoded by a start codon (e.g., a Methionine) at the N-terminus.

In one embodiment, a regulatory element is at least one stop codon. Therefore, in one embodiment, the invention relates to a nucleic acid sequence as set forth in SEQ ID NO:1, SEQ ID NO:3, SEQ ID NO:5, SEQ ID NO:7, SEQ ID NO:11 or SEQ ID NO:13 or a fragment or homolog thereof, operably linked to a nucleotide sequence comprising at least one stop codon at the 3′ terminus. In one embodiment, the nucleotide sequence is operably linked to two stop codons to increase the efficiency of translational termination.

In one embodiment, nucleic acid molecule can encode at least one peptide having an amino acid sequence set forth in SEQ ID NO:2, SEQ ID NO:4, SEQ ID NO: 6 or SEQ ID NO:8. In one embodiment, the nucleic acid molecule comprises the nucleotide sequence set forth in SEQ ID NO:1, SEQ ID NO:3, SEQ ID NO:5, SEQ ID NO:7, SEQ ID NO:11 or SEQ ID NO:13. In some embodiments, the sequence can be the nucleotide sequence having at least about 90%, 91%, 92%, 93%, 94%, 95%, 96%, 97%, 98%, 99% or 100% identity over an entire length of the nucleotide sequence set forth in SEQ ID NO:1, SEQ ID NO:3, SEQ ID NO:5, SEQ ID NO:7, SEQ ID NO:11 or SEQ ID NO:13. In other embodiments, sequence can be the nucleotide sequence that encodes the amino acid sequence having at least about 90%, 91%, 92%, 93%, 94%, 95%, 96%, 97%, 98%, 99%, or 100% identity over an entire length of the amino acid sequence set forth in SEQ ID NO:2, SEQ ID NO:4, SEQ ID NO: 6, SEQ ID NO:8, SEQ ID NO:12 or SEQ ID NO:14.

In some embodiments, the nucleic acid molecule comprises an RNA sequence that is a transcript from a DNA sequence having at least about 96%, 97%, 98%, 99% or 100% identity over an entire length of the nucleic acid sequence set forth in SEQ ID NO:1, SEQ ID NO:3, SEQ ID NO:5, SEQ ID NO:7, SEQ ID NO:11 or SEQ ID NO:13. In some embodiments, the nucleic acid molecule comprises an RNA sequence that encodes an amino acid sequence having at least about 96%, 97%, 98%, 99% or 100% identity over an entire length of the amino acid sequence set forth in SEQ ID NO:2, SEQ ID NO:4, SEQ ID NO: 6, SEQ ID NO:8, SEQ ID NO:12 or SEQ ID NO:14.

In one embodiment, the composition comprises a nucleic acid molecule comprising a nucleotide sequence which encodes at least one full length consensus FMDV-O immunogen. Nucleic acid sequences may comprise a sequence that encodes SEQ ID NO:2, SEQ ID NO:4, SEQ ID NO: 6, SEQ ID NO:8, SEQ ID NO:12 or SEQ ID NO:14. Nucleic acid sequence may comprise SEQ ID NO:1, SEQ ID NO:3, SEQ ID NO:5, SEQ ID NO:7, SEQ ID NO:11 or SEQ ID NO:13. Nucleic acid sequence may optionally comprise coding sequences that encode a signal peptide such as for example an IgE or IgG signal peptide.

The consensus-FMDV-O antigen can be a peptide having the amino acid sequence set forth in SEQ ID NO:2, SEQ ID NO:4, SEQ ID NO:6 or SEQ ID NO:8. In some embodiments, the antigen can have an amino acid sequence having at least about 90%, 91%, 92%, 93%, 94%, 95%, 96%, 97%, 98%, 99%, or 100% identity over an entire length of the amino acid sequence set forth in SEQ ID NO:2, SEQ ID NO:4, SEQ ID NO:6 or SEQ ID NO:8.

Immunogenic fragments of SEQ ID NO:2, SEQ ID NO:4, SEQ ID NO:6 or SEQ ID NO:8 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of the full length of SEQ ID NO:2, SEQ ID NO:4, SEQ ID NO:6 or SEQ ID NO:8. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:2, SEQ ID NO:4, SEQ ID NO:6 or SEQ ID NO:8, can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:2, SEQ ID NO:4, SEQ ID NO:6 or SEQ ID NO:8. Some embodiments relate to immunogenic fragments that have 96% homology to the immunogenic fragments of consensus protein sequences herein. Some embodiments relate to immunogenic fragments that have 97% homology to the immunogenic fragments of consensus protein sequences herein. Some embodiments relate to immunogenic fragments that have 98% homology to the immunogenic fragments of consensus protein sequences herein. Some embodiments relate to immunogenic fragments that have 99% homology to the immunogenic fragments of consensus protein sequences herein. In some embodiments, immunogenic fragments include a leader sequence, such as for example an immunoglobulin leader, such as the IgE leader. In some embodiments, immunogenic fragments are free of a leader sequence.

In one embodiment, an immunogenic fragment of a nucleic acid molecule encodes at least one immunodominant or sub-immunodominant epitope of a full length optimized consensus FMDV-O antigen.

Some embodiments relate to immunogenic fragments of SEQ ID NO:1, SEQ ID NO:3, SEQ ID NO:5 or SEQ ID NO:7 comprising at least 60%, at least 65%, at least 70%, at least 75%, at least 80%, at least 85%, at least 90%, at least 95%, at least 96%, at least 97%, at least 98% or at least 99% of the full length of SEQ ID NO:1, SEQ ID NO:3, SEQ ID NO:5 or SEQ ID NO:7. Immunogenic fragments can be at least 96%, at least 97% at least 98% or at least 99% homologous to fragments of SEQ ID NO:1, SEQ ID NO:3, SEQ ID NO:5 or SEQ ID NO:7. In some embodiments, immunogenic fragments include sequences that encode a leader sequence, such as for example an immunoglobulin leader, such as the IgE leader. In some embodiments, fragments are free of coding sequences that encode a leader sequence. In one embodiment, the nucleic acid sequence comprises an RNA sequence encoding a consensus FMDV-O immunogen sequence described herein. For example, nucleic acids may comprise an RNA sequence encoding one or more of SEQ ID NO:2, SEQ ID NO:4, SEQ ID NO: 6, SEQ ID NO:8, SEQ ID NO:12 or SEQ ID NO:14, a variant thereof, a fragment thereof or any combination thereof.

In some embodiments, the nucleic acid molecule includes a sequence that encodes for a FMDV-O antigen minus an IgE leader sequence on the N-terminal end of the coding sequence. In some embodiments, the DNA nucleic acid molecule further comprises an IgE leader sequence attached to an N-terminal end of the coding sequence and operably linked to the promoter.

The nucleic acid molecule can further include a polyadenylation sequence attached to the C-terminal end of the coding sequence. In one embodiment, the nucleic acid molecule is codon optimized.

In one embodiment, a fragment of a nucleic acid of the invention encodes a polypeptide capable of eliciting an immune response in a mammal substantially similar to that of the non-fragment for FMDV-O. The fragments may comprise at least 10%, at least 20%, at least 30%, at least 40%, at least 50%, at least 60%, at least 70%, at least 80%, at least 90% or at least 95% of a FMDV-O VP encoded by a nucleic acid sequence of SEQ ID NO:1, SEQ ID NO:3, SEQ ID NO:5, SEQ ID NO:7, SEQ ID NO:11 or SEQ ID NO:13. The DNA fragments may be 30 or more nucleotides in length, 45 or more, 60 or more, 75 or more, 90 or more, 120 or more, 150 or more, 180 or more, 210 or more, 240 or more, 270 or more, 300 or more, 360 or more, 420 or more, 480 or more, 540 or more, 600 or more, 660 or more, 720 or more, 780 or more, 840 or more, 900 or more, 960 or more, 1020 or more, 1080 or more, 1140 or more, 1200 or more, 1260 or more, 1320 or more, 1380 or more, 1440 or more, 1500 or more, 1560 or more, 1620 or more, 1680 or more, 1740 or more, 1800 or more, 1860 or more, 1820 or more, 1880 or more, 1940 or more, 2000 or more, 2100 or more, 2200 or more, 2300 or more, or 2310 or more nucleotides in length. Immunogenic fragments may comprise coding sequences for the immunoglobulin leader such as IgE or IgG sequences.

Immunogenic fragments may be fewer than 10 nucleotides, fewer than 20, fewer than 30, fewer than 40, fewer than 50, fewer than 60, fewer than 75, fewer than 90, fewer than 120, fewer than 150, fewer than 180, fewer than 210, fewer than 240, fewer than 270, fewer than 300, fewer than 360, fewer than 420, fewer than 480, fewer than 540, fewer than 600, fewer than 660, fewer than 720, fewer than 780, fewer than 840, fewer than 900, fewer than 960, fewer than 1020, fewer than 1080, fewer than 1140, fewer than 1200, fewer than 1260, fewer than 1320, fewer than 1380, fewer than 1440, fewer than 1500, fewer than 1560, fewer than 1620, fewer than 1680, or fewer than 1740 nucleotides, fewer than 1800, fewer than 1860, fewer than 1820, fewer than 1880, fewer than 1940, fewer than 2000, fewer than 2100, fewer than 2200, fewer than 2300, or fewer than 2310 nucleotides in length.

Coding sequences for these FMDV-O VP antigens, homologous antigens, fragments of FMDV-O VP antigens and fragments of homologous FMDV-O VP antigens may be used in constructs.

Table 1 provides exemplary consensus FMDV-O VP antigens and exemplary constructs comprising multiple VP antigens of the invention. Amino acid sequences for the consensus VP antigens and nucleic acid sequences encoding them are provided. The nucleic acid sequences encoding consensus VP antigens may be operably linked to one or more regulatory sequence. In one embodiment, one or more regulator sequences may be a start codon, at least one stop codon, or a combination thereof. Similarly, the amino acid sequences may be operably linked to one or more regulatory sequences or residues. In one embodiment, the amino acid sequences are operably linked to a methionine (M) at the N-terminus.

TABLE 1

| | | Amino |
|---|---|---|
| FMDV-O antigen | Nucleotide Sequence | Acid Sequence |
| VP1 | SEQ ID NO: 1 | SEQ ID NO: 2 |
| VP2 | SEQ ID NO: 3 | SEQ ID NO: 4 |
| VP3 | SEQ ID NO: 5 | SEQ ID NO: 6 |
| VP4 | SEQ ID NO: 7 | SEQ ID NO: 8 |
| VP4-VP2-VP3-VP1 | SEQ ID NO: 11 | SEQ ID NO: 12 |
| IgE-VP4-VP2-VP3-VP1-2A | SEQ ID NO: 13 | SEQ ID NO: 14 |

A proteolytic cleavage site can be present in between each of the consensus antigen sequences, such as the amino acid sequence: RGRKRRS (SEQ ID NO:15).

Provided herein is an antigen capable of eliciting an immune response in a mammal against one or more foot-and-mouth disease virus (FMDV) serotypes. The antigen may be a FMDV antigen comprising a consensus capsid protein VP1, VP2, VP3, VP4, a variant thereof, a fragment thereof or a combination thereof. The FMDV antigen may contain at least one antigenic epitope that may be effective against particular FMDV-O immunogens against which an immune response can be induced. The empty viral capsid proteins VP1-VP4 of the FMDV antigen provides an entire repertoire of immunogenic sites and epitopes present in an intact FMDV virus. The consensus FMDV antigen sequence may be derived from FMDV antigen sequences from a plurality of FMDV viruses of FMDV serotype O. The consensus FMDV antigen may comprise VP1, VP2, VP3, and VP4 FMDV serotype 0 consensus protein sequences, which may be a consensus VP1-VP4 protein. The consensus VP1-VP4 protein may comprise at least one protein cleavage site. The protein cleavage site may be present in between each of consensus VP1, VP2, VP3, and VP4 sequences of the consensus VP1-VP4 protein. Cleavage of the consensus VP1-VP4 protein may cleave the consensus VP1-VP4 protein to produce a consensus VP1-, a consensus VP2-, a consensus VP3-, and a consensus VP4 protein. The cleavage site can be present in between each of the consensus antigen sequences, such as the amino acid sequence: RGRKRRS (SEQ ID NO:15).

In some embodiments, coding sequences may encode fragments of the proteins described herein. In some embodiments, coding sequences encode proteins that are 20% of the consensus protein. In some embodiments, coding sequences encode proteins that are 30% of the consensus protein. In some embodiments, coding sequences encode proteins that are 40% of the consensus protein. In some embodiments, coding sequences encode proteins that are 50% of the consensus protein. In some embodiments, coding sequences encode proteins that are 60% of the consensus protein. In some embodiments, coding sequences encode proteins that are 70% of the consensus protein. In some embodiments, coding sequences encode proteins that are 85% of the consensus protein. In some embodiments, coding sequences encode proteins that are 90% of the consensus protein. In some embodiments, coding sequences encode proteins that are 95% of the consensus protein. In some embodiments, coding sequences encode proteins that are 96% of the consensus protein. In some embodiments, coding sequences encode proteins that are 97% of the consensus protein.

Additionally, coding sequences may encode proteins that are homologous to the proteins provided herein. In some embodiments, coding sequences encode proteins that are 80% homologous. In some embodiments, coding sequences encode proteins that are 90% homologous. In some embodiments, coding sequences encode proteins that are 95% homologous. In some embodiments, coding sequences encode proteins that are 96% homologous. In some embodiments, coding sequences encode proteins that are 97% homologous. In some embodiments, coding sequences encode proteins that are 98% homologous. In some embodiments, coding sequences encode proteins that are 99% homologous.

Additionally, coding sequences encode proteins that are fragments of proteins homologous to proteins described herein. In some embodiments, coding sequences encode proteins that are 20% of the homologous protein. In some embodiments, coding sequences encode proteins that are 30% of the homologous protein. In some embodiments, coding sequences encode proteins that are 40% of the homologous protein. In some embodiments, coding sequences encode proteins that are 50% of the homologous protein. In some embodiments, coding sequences encode proteins that are 60% of the homologous protein. In some embodiments, coding sequences encode proteins that are 70% of the homologous protein. In some embodiments, coding sequences encode proteins that are 80% of the homologous protein. In some embodiments, coding sequences encode proteins that are 90% of the homologous protein. In some embodiments, coding sequences encode proteins that are 95% of the homologous protein. In some embodiments, coding sequences encode proteins that are 96% of the homologous protein. In some embodiments, coding sequences encode proteins that are 97% of the homologous protein. In some embodiments, coding sequences encode proteins that are 98% of the homologous protein. In some embodiments, coding sequences encode proteins that are 99% of the homologous protein.

### Immunogenic Composition

Immunogenic compositions, such as vaccines, are provided comprising an optimized consensus sequence, an optimized consensus-encoded antigen, a fragment thereof, a variant thereof, or a combination thereof. The immunogenic composition can significantly induce an immune response of a subject administered with the immunogenic composition against the FMDV-O immunogen.

The immunogenic composition can be a DNA vaccine, an RNA vaccine, a peptide vaccine, or a combination vaccine. The vaccine can include an optimized consensus nucleotide sequence encoding an antigen. The nucleotide sequence can be DNA, RNA, cDNA, a variant thereof, a fragment thereof, or a combination thereof. The nucleotide sequence can also include additional sequences that encode linker, leader, or tag sequences that are linked to the antigen by a peptide bond. The peptide vaccine can include an antigen, a variant thereof, a fragment thereof, or a combination thereof. The combination DNA and peptide vaccine can include the above described optimized consensus nucleotide sequence and the encoded antigen.

The vaccine can be an attenuated live vaccine, a vaccine using recombinant vectors to deliver antigen, subunit vaccines, and glycoprotein vaccines, for example, but not limited, the vaccines described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,3 64; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference.

The vaccine of the present invention can have features required of effective vaccines such as being safe so that the vaccine itself does not cause illness or death; being protective against illness; inducing neutralizing antibody; inducing protective T cell responses; and providing ease of administration, few side effects, biological stability, and low cost per dose.

An immunogenic composition that can be used to elicit an immune response (humoral, cellular, or both) broadly against FMDV-O may comprise one or more coding sequences set forth above, i.e. a nucleic acid molecule comprising a nucleotide sequence that encodes one or more proteins VP1, VP2, VP3, VP4 and 2A from FMDV-O. In some embodiments the nucleic acid molecule may also comprise one or more nucleotide sequence encoding a proteolytic cleavage site.

The compositions may comprise a plurality of copies of a single nucleic acid molecule such a single plasmid, a plurality of copies of two or more different nucleic acid molecules such as two or more different plasmids. For example a composition may comprise plurality of two, three, four, five, six, seven, eight, nine or ten or more different nucleic acid molecules. Such compositions may comprise plurality of two, three, four, five, six, or more different plasmids.

Compositions may comprise nucleic acid molecules, such as plasmids, that collectively contain coding sequence for a single consensus FMDV-VP immunogen. Compositions may comprise nucleic acid molecules, such as plasmids, that collectively contain coding sequence for multiple consensus FMDV-VP immunogens.

Compositions may comprise nucleic acid sequence that encodes the combination of a consensus FMDV-O VP1 immunogen, a consensus FMDV-O VP2 immunogen, a consensus FMDV-O VP3 immunogen and a consensus FMDV-O VP4 immunogen. In one embodiment, each coding sequence for each consensus FMDV-VP immunogen is included on a separate plasmid. In an alternative embodiment, each coding sequence for each consensus FMDV-VP immunogen is included on a single plasmid. Accordingly, compositions that comprise nucleotide sequences that encode multiple consensus FMDV-VP immunogens may be on a single plasmid or on two or more separate plasmids.

Immunogenic compositions may be used to deliver nucleic acid molecules that encode one or more consensus FMDV-VP immunogen. The immunogenic composition may be provided to induce a therapeutic or prophylactic immune response.

Provided herein are pharmaceutical compositions according to the present invention which comprise about 1 nanogram to about 10 mg of nucleic acid. In some embodiments, pharmaceutical compositions according to the present invention comprise from between: 1) at least 10, 15, 20, 25, 30, 35, 40, 45, 50, 55, 60, 65, 70, 75, 80, 85, 90, 95 or 100 nanograms, or at least 1, 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995 or 1000 micrograms, or at least 1.5, 2, 2.5, 3, 3.5, 4, 4.5, 5, 5.5, 6, 6.5, 7, 7.5, 8, 8.5, 9, 9.5 or 10 mg or more; and 2) up to and including 15, 20, 25, 30, 35, 40, 45, 50, 55, 60, 65, 70, 75, 80, 85, 90, 95 or 100 nanograms, or up to and including 1, 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995, or 1000 micrograms, or up to and including 1.5, 2, 2.5, 3, 3.5, 4, 4.5, 5, 5.5, 6, 6.5, 7, 7.5, 8, 8.5, 9, 9.5 or 10 mg. In some embodiments, pharmaceutical compositions according to the present invention comprise about 5 nanogram to about 10 mg of nucleic acid. In some embodiments, pharmaceutical compositions according to the present invention comprise about 25 nanogram to about 5 mg of nucleic acid. In some embodiments, the pharmaceutical compositions contain about 50 nanograms to about 1 mg of nucleic acid. In some embodiments, the pharmaceutical compositions contain about 0.1 to about 500 micrograms of nucleic acid. In some embodiments, the pharmaceutical compositions contain about 1 to about 350 micrograms of nucleic acid. In some embodiments, the pharmaceutical compositions contain about 5 to about 250 micrograms of nucleic acid. In some embodiments, the pharmaceutical compositions contain about 10 to about 200 micrograms of nucleic acid. In some embodiments, the pharmaceutical compositions contain about 15 to about 150 micrograms of nucleic acid. In some embodiments, the pharmaceutical compositions contain about 20 to about 100 micrograms of nucleic acid. In some embodiments, the pharmaceutical compositions contain about 25 to about 75 micrograms of nucleic acid. In some embodiments, the pharmaceutical compositions contain about 30 to about 50 micrograms of nucleic acid. In some embodiments, the pharmaceutical compositions contain about 35 to about 40 micrograms of nucleic acid. In some embodiments, the pharmaceutical compositions contain about 100 to about 200 microgram nucleic acid. In some embodiments, the pharmaceutical compositions comprise about 10 microgram to about 100 micrograms of nucleic acid. In some embodiments, the pharmaceutical compositions comprise about 20 micrograms to about 80 micrograms of nucleic acid. In some embodiments, the pharmaceutical compositions comprise about 25 micrograms to about 60 micrograms of nucleic acid. In some embodiments, the pharmaceutical compositions comprise about 30 nanograms to about 50 micrograms of nucleic acid. In some embodiments, the pharmaceutical compositions comprise about 35 nanograms to about 45 micrograms of nucleic acid. In some preferred embodiments, the pharmaceutical compositions contain about 0.1 to about 500 micrograms of nucleic acid. In some preferred embodiments, the pharmaceutical compositions contain about 1 to about 350 micrograms of nucleic acid. In some preferred embodiments, the pharmaceutical compositions contain about 25 to about 250 micrograms of nucleic acid. In some preferred embodiments, the pharmaceutical compositions contain about 100 to about 200 microgram nucleic acid.

The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

In one embodiment, the pharmaceutical composition is a vaccine. The vaccine may be a nucleic acid vaccine. The nucleic acid vaccine may comprise one or more nucleic acid sequences that encode one or more of consensus FMDV-O VP antigens. The nucleic acid vaccine may comprise a plurality of the same or different plasmids comprising nucleic acid coding sequences for one or more of consensus FMDV-O VP antigens. When the nucleic acid vaccine comprises coding sequences of more than one consensus FMDV-O VP antigens all such sequences may be present on a single plasmid, or each such sequences may be present on a different plasmid.

Nucleic acid vaccines are disclosed in U.S. Pat. Nos. 5,593,972, 5,739,118, 5,817,637, 5,830,876, 5,962,428, 5,981,505, 5,580,859, 5,703,055, and 5,676,594, which are incorporated herein fully by reference. The nucleic acid vaccine can further comprise elements or reagents that inhibit it from integrating into the chromosome. The vaccine can be an RNA of the one or more consensus FMDV-O VP antigens. The RNA vaccine can be introduced into the cell.

The vaccine can be a recombinant vaccine comprising the genetic construct or antigen described above. The vaccine can also comprise one or more consensus FMDV-O VP antigens in the form of one or more protein subunits, or one or more attenuated viral particles comprising one or more consensus antigens. The attenuated vaccine can be attenuated live vaccines, killed vaccines and vaccines that use recombinant vectors to deliver foreign genes that encode one or more consensus FMDV-O VP antigens, and well as subunit and protein vaccines. Examples of attenuated live vaccines, those using recombinant vectors to deliver antigens, subunit vaccines and glycoprotein vaccines are described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,3 64; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference. Vaccines may comprise plasmids in combination with other vaccine components such as FMDV proteins or expression vectors encoding proteins.

The immunogenic composition provided may be used to induce immune responses including therapeutic or prophylactic immune responses. Antibodies and/or killer T cells may be generated which are directed to the consensus FMDV-O VP antigen. Such antibodies and cells may be isolated.

The pharmaceutically acceptable excipient may be one or more adjuvants. An adjuvant may be other genes that are expressed from the same or from an alternative plasmid or are delivered as proteins in combination with the plasmid above in the immunogenic composition. The one or more adjuvants may be proteins and/or nucleic acid molecules that encode proteins selected from the group consisting of: CCL20, α-interferon (IFN-α), (β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15 including IL-15 having the signal sequence or coding sequence that encodes the signal sequence deleted and optionally including a different signal peptide such as that from IgE or coding sequence that encodes a difference signal peptide such as that from IgE, IL-28, MHC, CD80, CD86, IL-1, IL-2, IL-4, IL-5, IL-6, IL-10, IL-18, MCP-1, MIP-1α, MIP-1β, IL-8, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or a combination thereof. In some embodiments adjuvant may be one or more proteins and/or nucleic acid molecules that encode proteins selected from the group consisting of: CCL-20, IL-12, IL-15, IL-28, CTACK, TECK, MEC or RANTES. Examples of IL-12 constructs and sequences are disclosed in PCT application no. PCT/US1997/019502 and corresponding U.S. application Ser. No. 08/956,865, and U.S. Provisional Application Ser. No 61/569600 filed Dec. 12, 2011, which are each incorporated herein by reference. Examples of IL-15 constructs and sequences are disclosed in PCT application no. PCT/US04/18962 and corresponding U.S. application Ser. No. 10/560,650, and in PCT application no. PCT/US07/00886 and corresponding U.S. application Ser. No. 12/160,766, and in PCT application no. PCT/US10/048827, which are each incorporated herein by reference. Examples of IL-28 constructs and sequences are disclosed in PCT application no. PCT/US09/039648 and corresponding U.S. application Ser. No. 12/936,192, which are each incorporated herein by reference. Examples of RANTES and other constructs and sequences are disclosed in PCT application no. PCT/US1999/004332 and corresponding U.S. application Ser. No. and 09/622452, which are each incorporated herein by reference. Other examples of RANTES constructs and sequences are disclosed in PCT application no. PCT/US11/024098, which is incorporated herein by reference. Examples of RANTES and other constructs and sequences are disclosed in PCT application no. PCT/US1999/004332 and corresponding U.S. application Ser. No. 09/622452, which are each incorporated herein by reference. Other examples of RANTES constructs and sequences are disclosed in PCT application no. PCT/US11/024098, which is incorporated herein by reference. Examples of chemokines CTACK, TECK and MEC constructs and sequences are disclosed in PCT application no. PCT/US2005/042231 and corresponding U.S. application Ser. No. 11/719,646, which are each incorporated herein by reference. Examples of OX40 and other immunomodulators are disclosed in U.S. application Ser. No. 10/560,653, which is incorporated herein by reference. Examples of DR5 and other immunomodulators are disclosed in U.S. application Ser. No. 09/622452, which is incorporated herein by reference.

The transfection facilitating agent is a polyanion, polycation, including poly-L-glutamate (LGS), or lipid. The transfection facilitating agent is poly-L-glutamate, and more preferably, the poly-L-glutamate is present in the immunogenic composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the nucleic acid plasmid immunogenic composition s may also include a transfection facilitating agent such as lipids, liposomes, including lecithin liposomes or other liposomes known in the art, as a nucleic acid-liposome mixture (see for example W09324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid. Concentration of the transfection agent in the immunogenic composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient may be an adjuvant. The adjuvant may be other genes that are expressed in alternative plasmid or are delivered as proteins in combination with the plasmid above in the immunogenic composition. The adjuvant may be selected from the group consisting of: α-interferon(IFN-α), (β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80,CD86 including IL-15 having the signal sequence deleted and optionally including the signal peptide from IgE. The adjuvant may be IL-12, IL-15, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes which may be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The immunogenic composition may further comprise a genetic vaccine facilitator agent as described in U.S. Ser. No. 021,579 filed Apr. 1, 1994, which is fully incorporated by reference.

The immunogenic composition may be formulated according to the mode of administration to be used. An injectable immunogenic composition pharmaceutical composition may be sterile, pyrogen free and particulate free. An isotonic formulation or solution may be used. Additives for isotonicity may include sodium chloride, dextrose, mannitol, sorbitol, and lactose. The immunogenic composition may comprise a vasoconstriction agent. The isotonic solutions may include phosphate buffered saline.

Immunogenic composition may further comprise stabilizers including gelatin and albumin. The stabilizing may allow the formulation to be stable at room or ambient temperature for extended periods of time such as LGS or polycations or polyanions to the immunogenic composition formulation.

The immunogenic composition may be stable at room temperature (25° C.) for more than 1 week, in some embodiments for more than 2 weeks, in some embodiments for more than 3 weeks, in some embodiments for more than 4 weeks, in some embodiments for more than 5 weeks, and in some embodiments for more than 6 weeks. In some embodiments, the vaccine is stable for more than one month, more than 2 months, more than 3 months, more than 4 months, more than 5 months, more than 6 months, more than 7 months, more than 8 months, more than 9 months, more than 10 months, more than 11 months, or more than 12 months. In some embodiments, the vaccine is stable for more than 1 year, more than 2 years, more than years, or more than 5 years. In one embodiment, the immunogenic composition is stable under refrigeration (2-8° C.). Accordingly, in one embodiment, the immunogenic composition does not require frozen cold-chain. An immunogenic composition is stable if it retains its biological activity for a sufficient period to allow its intended use (e.g., to generate an immune response in a subject). For example, for immunogenic compositions that are to be stored, shipped, etc., it may be desired that the immunogenic compositions remain stable for months to years.

### Immune Response

The immunogenic composition can induce an immune response in the subject administered the composition. The induced immune response can be specific for a FMDV-O antigen. The induced immune response can be reactive with a FMDV-O antigen related to the optimized consensus-encoded antigen. In various embodiments, related antigens include antigens having amino acid sequences having at least 90%, at least 91%, at least 92%, at least 93%, at least 94%, at least 95%, at least 96%, at least 97%, at least 98%, at least 99%, or 100% homology to the amino acid sequence of the optimized consensus-encoded antigen. In various embodiments, related antigens include antigens encoded by nucleotide sequences having at least 90%, at least 91%, at least 92%, at least 93%, at least 94%, at least 95%, at least 96%, at least 97%, at least 98%, at least 99%, or 100% homology to the optimized consensus nucleotide sequences disclosed herein.

The immunogenic composition can induce a humoral immune response in the subject administered the immunogenic composition. The induced humoral immune response can be specific for a FMDV-O antigen. The induced humoral immune response can be reactive with the FMDV-O antigen related to the optimized consensus-encoded antigen. The humoral immune response can be induced in the subject administered the immunogenic composition by about 1.5-fold to about 16-fold, about 2-fold to about 12-fold, or about 3-fold to about 10-fold. The humoral immune response can be induced in the subject administered the immunogenic composition by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to a subject not administered the immunogenic composition or a subject administered a non-optimized FMDV-O antigen.

The humoral immune response induced by the immunogenic composition can include an increased level of neutralizing antibodies associated with the subject administered the immunogenic composition as compared to a subject not administered the immunogenic composition. The neutralizing antibodies can be specific for a FMDV-O antigen related to the optimized consensus-encoded antigen. The neutralizing antibodies can be reactive with the FMDV-O antigen genetically related to the optimized consensus antigen. The neutralizing antibodies can provide protection against and/or treatment of tumor growth, metastasis or tumor associated pathologies in the subject administered the immunogenic composition.

The humoral immune response induced by the immunogenic composition can include an increased level of IgG antibodies associated with the subject administered the immunogenic composition as compared to a subject not administered the immunogenic composition. These IgG antibodies can be specific for the FMDV-O antigen genetically related to the optimized consensus antigen. These IgG antibodies can be reactive with the FMDV-O antigen genetically related to the optimized consensus antigen. The level of IgG antibody associated with the subject administered the immunogenic composition can be increased by about 1.5-fold to about 16-fold, about 2-fold to about 12-fold, or about 3-fold to about 10-fold as compared to the subject not administered the immunogenic composition. The level of IgG antibody associated with the subject administered the immunogenic composition can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to a subject not administered the immunogenic composition or a subject administered a non-optimized FMDV-O antigen.

The immunogenic composition can induce a cellular immune response in the subject administered the immunogenic composition. The induced cellular immune response can be specific for a FMDV-O antigen related to the optimized consensus-encoded antigen. The induced cellular immune response can be reactive to the FMDV-O antigen related to the optimized consensus-encoded antigen. The induced cellular immune response can include eliciting a CD8⁺ T cell response. The elicited CD8⁺ T cell response can be reactive with the FMDV-O antigen genetically related to the optimized consensus antigen. The elicited CD8⁺ T cell response can be polyfunctional. The induced cellular immune response can include eliciting a CD8⁺ T cell response, in which the CD8⁺ T cells produce interferon-gamma (IFN-γ), tumor necrosis factor alpha (TNF-α), interleukin-2 (IL-2), or a combination of IFN-γ and TNF-α.

The induced cellular immune response can include an increased CD8⁺ T cell response associated with the subject administered the immunogenic composition as compared to the subject not administered the immunogenic composition. The CD8⁺ T cell response associated with the subject administered the immunogenic composition can be increased by about 2-fold to about 30-fold, about 3-fold to about 25-fold, or about 4-fold to about 20-fold as compared to the subject not administered the immunogenic composition. The CD8⁺ T cell response associated with the subject administered the immunogenic composition can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to a subject not administered the immunogenic composition or a subject administered a non-optimized FMDV-O antigen.

The induced cellular immune response can include an increased frequency of CD107a/IFNγ/T-bet triple-positive CD8 T cells that are reactive against the FMDV-O antigen. The frequency of CD107a/IFNγ/T-bet triple-positive CD8 T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to a subject not administered the immunogenic composition or a subject administered a non-optimized FMDV-O antigen.

The induced cellular immune response can include an increased frequency of CD107a/IFNγdouble-positive CD8 T cells that are reactive against the FMDV-O antigen. The frequency of CD107a/IFNγ double-positive CD8 T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to a subject not administered the immunogenic composition or a subject administered a non-optimized FMDV-O antigen.

The cellular immune response induced by the immunogenic composition can include eliciting a CD4⁺ T cell response. The elicited CD4⁺ T cell response can be reactive with the FMDV-O antigen genetically related to the optimized consensus antigen. The elicited CD4⁺ T cell response can be polyfunctional. The induced cellular immune response can include eliciting a CD4⁺ T cell response, in which the CD4⁺ T cells produce IFN-γ, TNF-α, IL-2, or a combination of IFN-γ and TNF-α.

The induced cellular immune response can include an increased frequency of CD4⁺ T cells that produce IFN-γ. The frequency of CD4⁺IFN-γ⁺ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to a subject not administered the immunogenic composition or a subject administered a non-optimized FMDV-O antigen.

The induced cellular immune response can include an increased frequency of CD4⁺ T cells that produce TNF-α. The frequency of CD4⁺TNF-α⁺ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to a subject not administered the immunogenic composition or a subject administered a non-optimized FMDV-O antigen.

The induced cellular immune response can include an increased frequency of CD4⁺ T cells that produce both IFN-γ and TNF-α. The frequency of CD4⁺IFN-γ⁺TNF-α⁺ associated with the subject administered the immunogenic composition can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to a subject not administered the immunogenic composition or a subject administered a non-optimized FMDV-O antigen.

The immunogenic composition can further induce an immune response when administered to different tissues such as the muscle or skin. The immunogenic composition can further induce an immune response when administered via electroporation, or injection, or subcutaneously, or intramuscularly.

### Vector

The nucleic acid sequence construct described above can be placed in one or more vectors. Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.).

Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

(1) Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

(2) Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, Calif.), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif), which may be used for protein production in Escherichia coli (E.coli). The plasmid may also be pYES2 (Invitrogen, San Diego, Calif), which may be used for protein production in Saccharomyces cerevisiae strains of yeast. The plasmid may also be of the MAXBAC™ complete baculovirus expression system (Invitrogen, San Diego, Calif), which may be used for protein production in insect cells. The plasmid may also be pcDNAI or pcDNA3 (Invitrogen, San Diego, Calif), which may be used for protein production in mammalian cells such as Chinese hamster ovary (CHO) cells.

(3) RNA

In one embodiment, the nucleic acid is an RNA molecule. In one embodiment, the RNA molecule is transcribed from a DNA sequence described herein. For example, in some embodiments, the RNA molecule is encoded by a DNA sequence at least 90% homologous to one of SEQ ID NO:1, SEQ ID NO:3, SEQ ID NO:5, SEQ ID NO:7, SEQ ID NO:11 or SEQ ID NO:13. In another embodiment, the nucleotide sequence comprises an RNA sequence transcribed by a DNA sequence encoding a polypeptide sequence of SEQ ID NO:2, SEQ ID NO:4, SEQ ID NO:6, SEQ ID NO:8, SEQ ID NO:12, or SEQ ID NO:14 or a variant thereof or a fragment thereof. Accordingly, in one embodiment, the invention provides an RNA molecule encoding one or more of the DMAbs. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A

RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

(4) Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be perM2. The LEC can be perNP. perNP and perMR can be derived from pNP (Puerto Rico/34) and pM2 (New Caledonia/99), respectively.

(5) Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

(6) Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### Multiple Vectors

The immunogenic composition may comprise a plurality of copies of a single nucleic acid molecule such a single plasmid, or a plurality of copies of two or more different nucleic acid molecules such as two or more different plasmids. For example an immunogenic composition may comprise plurality of two, three, four, five, six, seven, eight, nine or ten or more different nucleic acid molecules. Such compositions may comprise plurality of two, three, four, five, six, or more different plasmids.

Immunogenic compositions may comprise nucleic acid molecules, such as plasmids, that collectively contain coding sequence for a FMDV-O antigen. Immunogenic compositions may comprise nucleic acid molecules, such as plasmids, that collectively contain coding sequence for multiple antigens. In one embodiment, the antigens are a FMDV-O antigen and one or more additional cancer antigen. Immunogenic compositions may comprise nucleic acid molecules, such as plasmids, that collectively contain coding sequence for one or more antigen and one or more cancer antigen.

Plasmids may comprise one or more coding sequences encoding one or more consensus FMDV VP antigens selected from consensus VP1, VP2, VP3, and VP4 from FMDV-O.

In some embodiments, a plasmid comprises coding sequences for two or more consensus FMDV VP antigens selected from consensus VP1, VP2, VP3, and VP4 from FMDV-O.

### Methods

Also provided herein is a method of treating, protecting against, and/or preventing disease in a subject in need thereof by administering the immunogenic composition to the subject. Administration of the immunogenic composition to the subject can induce or elicit an immune response in the subject. The induced immune response can be used to treat, prevent, and/or protect against disease, for example, foot-and-mouth disease virus infection or foot-and-mouth disease.

Provided herein is a method for delivering the immunogenic composition for providing genetic constructs and proteins of the FMDV antigen which comprise epitopes that make them particular effective against immunogens of FMDV against which an immune response can be induced. The method of delivering the immunogenic composition or vaccination may be provided to induce a therapeutic and prophylactic immune response.

The vaccination process may generate in the mammal an immune response against FMDV-O. The vaccination process may generate in the mammal an immune response against a plurality of FMDV serotypes. The immunogenic composition may be delivered to an individual to modulate the activity of the mammal's immune system and enhance the immune response. The delivery of the immunogenic composition may be the transfection of the FMDV antigen as a nucleic acid molecule that is expressed in the cell and delivered to the surface of the cell upon which the immune system recognized and induces a cellular, humoral, or cellular and humoral response. The delivery of the immunogenic composition may be use to induce or elicit and immune response in mammals against one or more FMDV serotype viruses by administering to the mammals the immunogenic composition as discussed above.

Upon delivery of the immunogenic composition and plasmid into the cells of the mammal, the transfected cells will express and secrete consensus capsids for each of the plasmids injected from the immunogenic composition. These secreted capsid proteins will be recognized as foreign by the immune system and antibodies will be made against them. These antibodies will be maintained by the immune system and allow for rapid clearing of subsequent FMDV challenge.

The immunogenic composition may be administered to a mammal to elicit an immune response in a mammal. The mammal may be human, primate, non-human primate, cow, cattle, sheep, goat, antelope, bison, water buffalo, bison, bovids, deer, hedgehogs, elephants, llama, alpaca, mice, rats, and chicken. The induced immune response can include an induced humoral immune response and/or an induced cellular immune response. The humoral immune response can be induced by about 1.5-fold to about 16-fold, about 2-fold to about 12-fold, or about 3-fold to about 10-fold. The induced humoral immune response can include IgG antibodies and/or neutralizing antibodies that are reactive to the antigen. The induced cellular immune response can include a CD8⁺ T cell response, which is induced by about 2-fold to about 30-fold, about 3-fold to about25-fold, or about 4-fold to about 20-fold.

The immunogenic composition dose can be between 1 μg to 10 mg active component/kg body weight/time, and can be 20 μg to 10 mg component/kg body weight/time. The immunogenic composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of immunogenic composition doses for effective treatment can be 1, 2, 3, 4, 5, 6, 7, 8, 9, or 10.

The immunogenic composition can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration.

The immunogenic composition can be administered prophylactically or therapeutically. In prophylactic administration, the immunogenic compositions can be administered in an amount sufficient to induce an immune response. In therapeutic applications, the immunogenic compositions are administered to a subject in need thereof in an amount sufficient to elicit a therapeutic effect. An amount adequate to accomplish this is defined as “therapeutically effective dose.” Amounts effective for this use will depend on, e.g., the particular composition of the immunogenic composition regimen administered, the manner of administration, the stage and severity of the disease, the general state of health of the subject, and the judgment of the prescribing physician.

The immunogenic composition can be administered by methods well known in the art as described in Donnelly et al. (Ann. Rev. Immunol. 15:617-648 (1997)); Feigner et al. (U.S. Pat. No. 5,580,859, issued Dec. 3, 1996); Feigner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the immunogenic composition can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The immunogenic composition can be delivered via a variety of routes. Typical delivery routes include parenteral administration, e.g., intradermal, intramuscular or subcutaneous delivery. Other routes include oral administration, intranasal, and intravaginal routes. For the DNA of the immunogenic composition in particular, the immunogenic composition can be delivered to the interstitial spaces of tissues of an individual (Feigner et al., U.S. Pat. Nos. 5,580,859 and 5,703,055, the contents of all of which are incorporated herein by reference in their entirety). The immunogenic composition can also be administered to muscle, or can be administered via intradermal or subcutaneous injections, or transdermally, such as by iontophoresis. Epidermal administration of the immunogenic composition can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The immunogenic composition can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the immunogenic composition.

The immunogenic composition can be a liquid preparation such as a suspension, syrup or elixir. The immunogenic composition can also be a preparation for parenteral, subcutaneous, intradermal, intramuscular or intravenous administration (e.g., injectable administration), such as a sterile suspension or emulsion.

The immunogenic composition can be incorporated into liposomes, microspheres or other polymer matrices (Felgner et al., U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

### Combination Treatments

The immunogenic composition may be administered in combination with other proteins or genes. Exemplary genes include, but are not limited to genes encoding α-interferon, γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80,CD86 including IL-15 having the signal sequence deleted and optionally including the signal peptide from IgE, IL-12, IL-15, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or combinations thereof. The immunogenic composition may also be administered in combination with CTACK protein, TECK protein, MEC protein or functional fragments thereof.

The immunogenic composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The immunogenic composition may be administered by traditional syringes, needleless injection devices, “microprojectile bombardment gone guns”, or other physical methods such as electroporation (“EP”), “hydrodynamic method”, or ultrasound.

The plasmid of the vaccine may be delivered to the mammal by several well-known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The FMDV antigen may be delivered via DNA injection and along with in vivo electroporation.

### Electroporation

Administration of the immunogenic composition via electroporation of the plasmids of the immunogenic composition may be accomplished using electroporation devices that can be configured to deliver to a desired tissue of a mammal a pulse of energy producing a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electronporation may be accomplished using the VGXP Cellectra™ system to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the immunogenic compositions of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the immunogenic compositions include those provided in co-pending and co-owned U.S. patent application, Ser. No. 11/874072, filed Oct. 17, 2007, which claims the benefit under 35 USC 119(e) to U.S. Provisional Applications Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No.6,697,669 issued Feb. 24, 2004, which concerns delivery of nucleic acid using any of a variety of devices, and U.S. Pat. No. 7,328,064 issued Feb. 5, 2008, drawn to method of injecting nucleic acid are contemplated herein. The above-patents are incorporated by reference in their entirety.

### Generation of Antigens In Vitro and Ex Vivo

In one embodiment, the optimized consensus FMDV-O antigen is generated in vitro or ex vivo. For example, in one embodiment, a nucleic acid encoding an optimized consensus FMDV-O antigen can be introduced and expressed in an in vitro or ex vivo cell.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### EXAMPLES

### Example 1

### DNA Vaccine Targeting FMDV-O

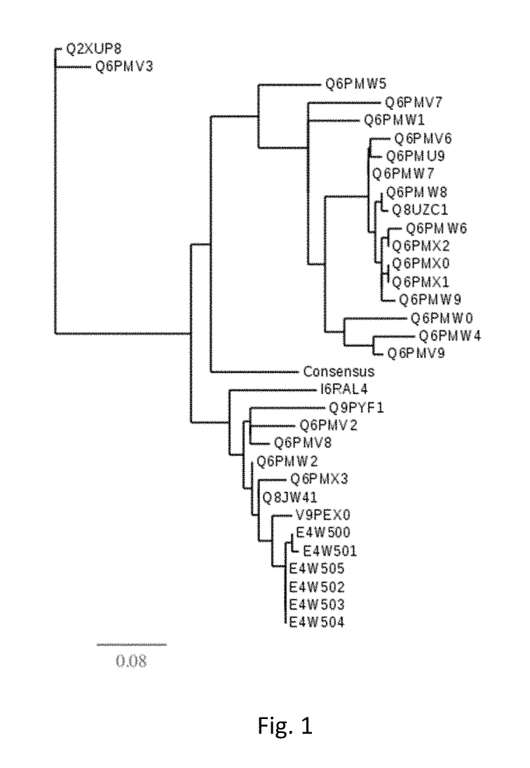

As set out in FIGS. 1-7, a construct encoding consensus FMDV-O VP1-VP4 has been made and tested.

FIG. 1 shows the relationship of the consensus VP1 to divergent VP1 from various FMDV-O strains.

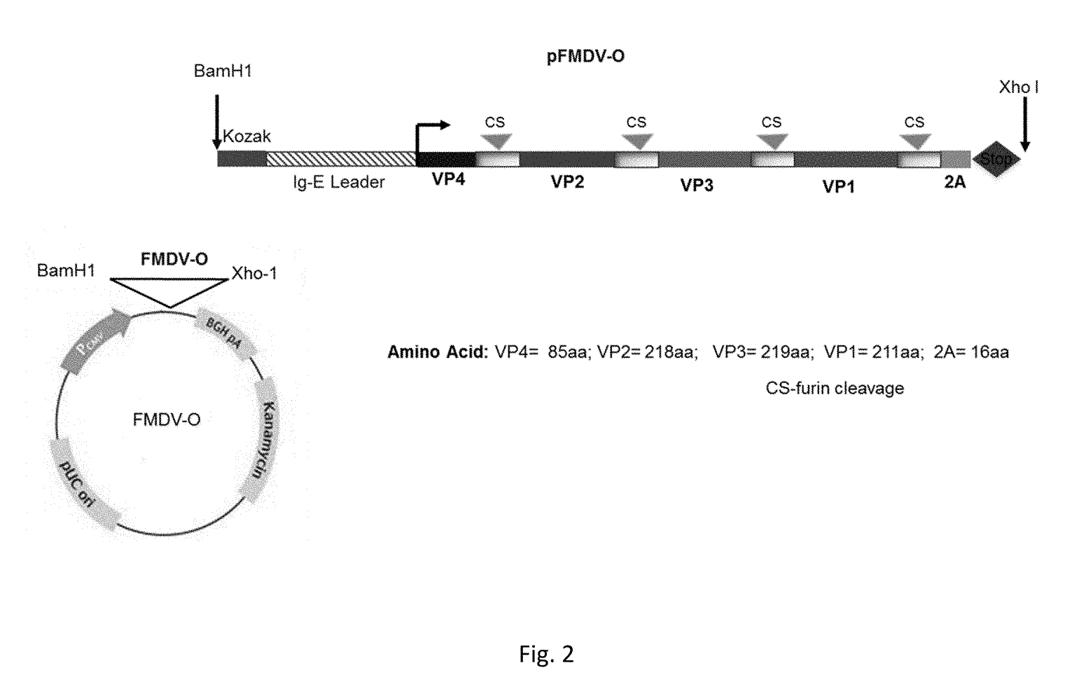

FIG. 2 shows schematic representation of a FMDV-O DNA construct, indicating that the insert is cloned into an expression plasmid. A plasmid map of pFMDV-O is shown. The IgE leader shown is indicated to be optional or may substituted with a different leader. The 2A sequence is indicated as optional and the furin cleavage site (rgrkrrs—SEQ ID NO:15) is indicated as being substitutable.

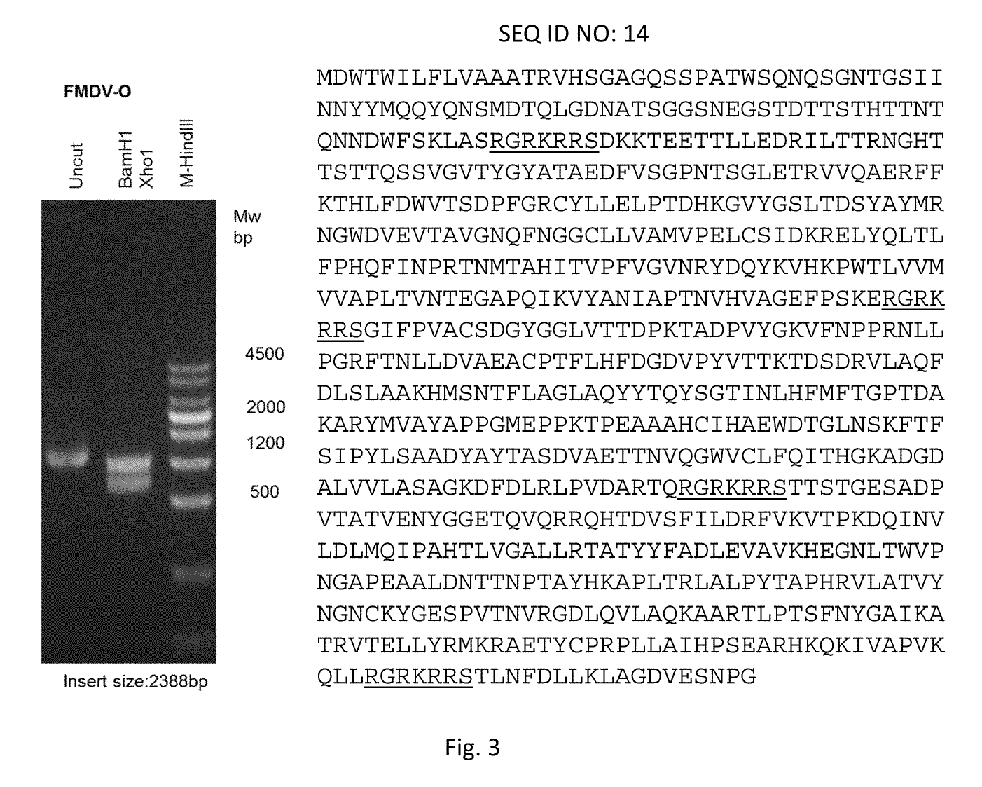

FIG. 3 shows a stained gel showing cloning of pFMDV-O. This data shows that the inserts have been properly incorporated into the expression plasmid. FIG. 3 also shows the amino acid sequence for FMDV-O. The sequence included the IgE leader sequence at the N terminus shaded and the proteolytic cleavage sites (underlined.) The VP4 sequence is between the IgE leader and first proteolytic cleavage site. Between the first proteolytic cleavage site and the second proteolytic cleavage site is the coding sequence of VP2. Between the second proteolytic cleavage site and the third proteolytic cleavage site is the coding sequence of VP3. Between the third proteolytic cleavage site and the fourth proteolytic cleavage site is the coding sequence of VP1. The 2A sequence is between the last (fourth) proteolytic cleavage site and stop.

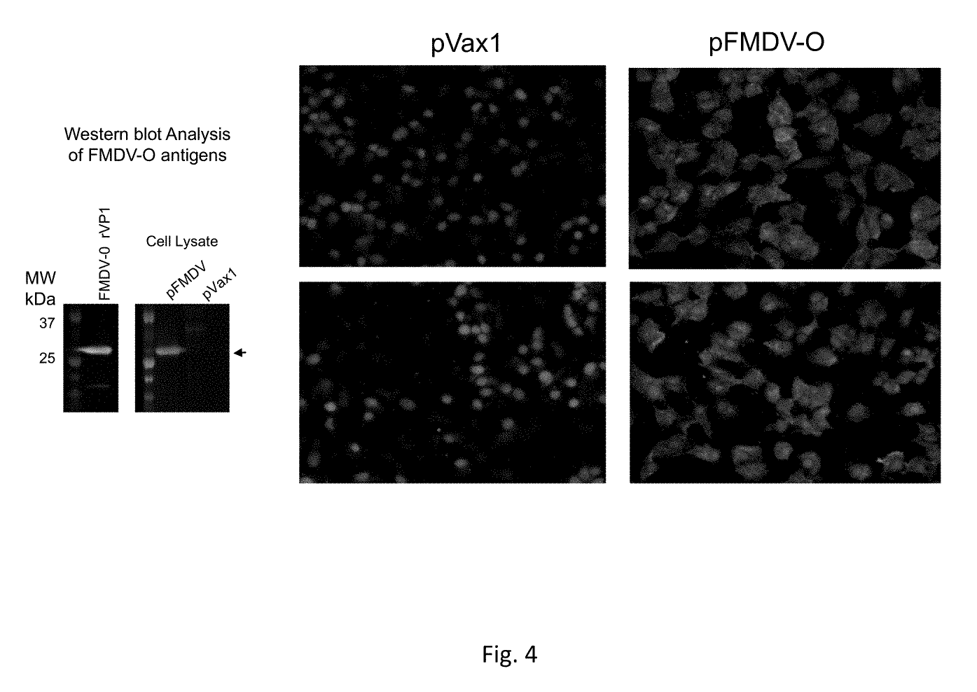

FIG. 4 shows experimental results of protein expression. Western blots of protein on SDS gels and immunofluorescent assays were used to confirm protein expression from samples transformed with pFMDV-O.

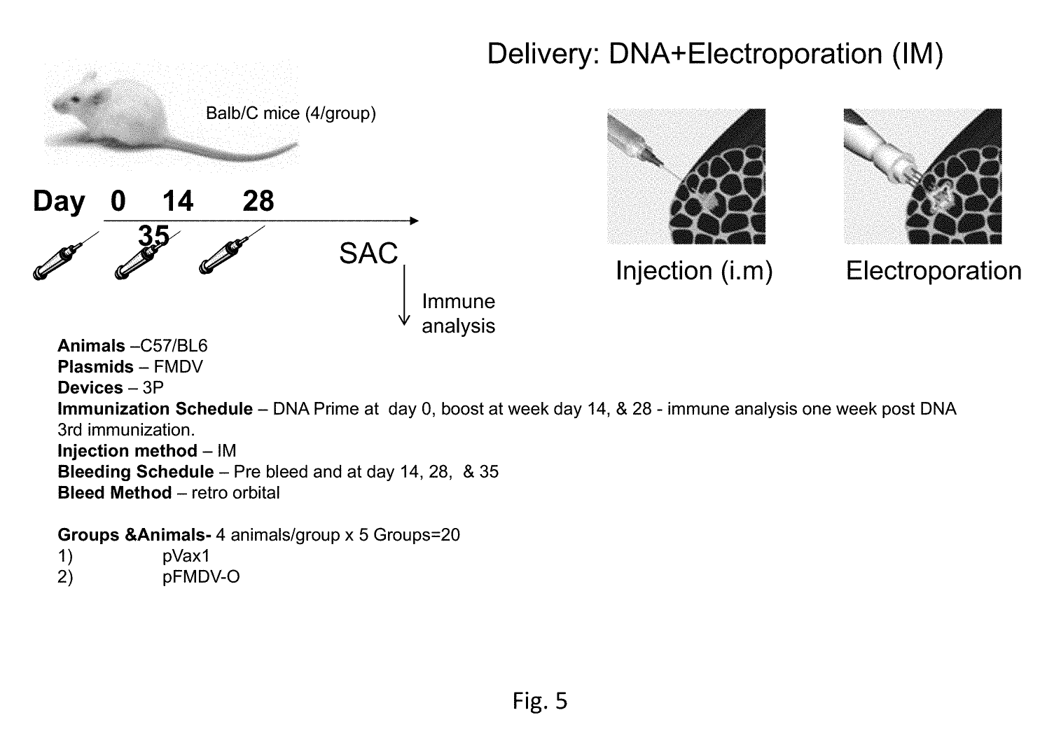

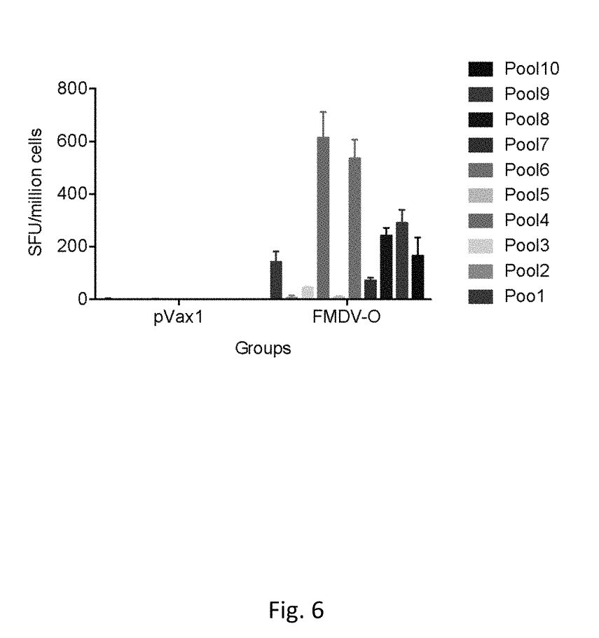

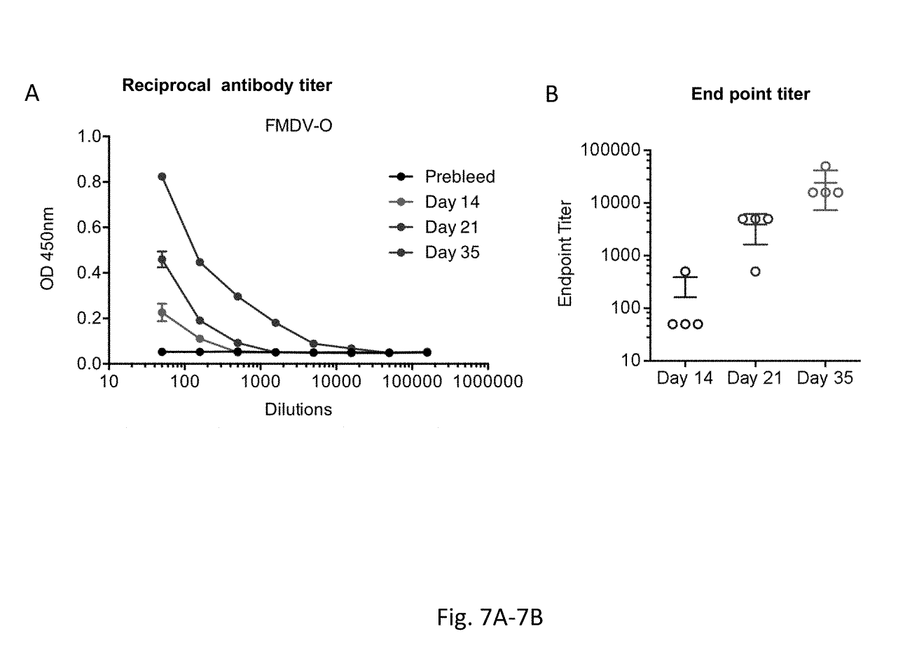

FIG. 5 shows an experimental protocol of immunization experiments using electroporation to evaluate immune responses following administration of 1) a control plasmid or 2) pFMDV-O. FIG. 6 shows data of cellular immune responses elicited by FMDV-O vaccination. FIG. 7 shows data of antibody induction in mice elicited by FMDV-O vaccination. Seroconversion was observed in all immunized animals. Humoral responses appear most potent. Characterization of T cells shows reasonable levels of immune reactivity was induced by the immunogenic compositions.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A nucleic acid molecule comprising a nucleotide sequence encoding one or more foot-and-mouth disease virus serotype O (FMDV-O) consensus viral capsid protein (VP) antigens.

2. The nucleic acid molecule of claim 1 comprising one or more nucleotide sequence selected from the group consisting of:
a) a nucleotide sequence encoding a FMDV-O antigen selected from the group consisting of SEQ ID NO:2, SEQ ID NO:4, SEQ ID NO:6 and SEQ ID NO:8; and
b) a nucleotide sequence encoding a FMDV-O antigen selected from the group consisting of a sequence having at least 95% homology to the amino acid sequence set forth in SEQ ID NO:2, a sequence having at least 95% homology to the amino acid sequence set forth in SEQ ID NO:4, a sequence having at least 95% homology to the amino acid sequence set forth in SEQ ID NO:6, and a sequence having at least 95% homology to the amino acid sequence set forth in SEQ ID NO:8.

3. The nucleic acid molecule of claim 2 comprising one or more nucleotide sequence selected from the group consisting of:
a) a nucleotide sequence selected from the group consisting of SEQ ID NO:1, SEQ ID NO:3, SEQ ID NO:5 and SEQ ID NO:7; and
b) a nucleotide sequence selected from the group consisting of a sequence having at least 95% homology to the nucleotide sequence set forth in SEQ ID NO:1, a sequence having at least 95% homology to the nucleotide sequence set forth in SEQ ID NO:3, a sequence having at least 95% homology to the nucleotide sequence set forth in SEQ ID NO:5, and a sequence having at least 95% homology to the nucleotide sequence set forth in SEQ ID NO:7.

4. The nucleic acid molecule of claim 2 comprising nucleotide sequences encoding FMDV-O VP1, VP2, VP3 and VP4 selected from the group consisting of:
a) nucleotide sequences encoding amino acid sequences as set forth in SEQ ID NO:2, SEQ ID NO:4, SEQ ID NO:6 and SEQ ID NO:8; and
b) nucleotide sequences encoding an amino acid sequence having at least 95% homology to the amino acid sequence set forth in SEQ ID NO:2, an amino acid sequence having at least 95% homology to the amino acid sequence set forth in SEQ ID NO:4, an amino acid sequence having at least 95% homology to the amino acid sequence set forth in SEQ ID NO:6 and an amino acid sequence having at least 95% homology to the amino acid sequence set forth in SEQ ID NO:8.

5. The nucleic acid molecule of claim 4 comprising nucleotide sequences selected from the group consisting of:
a) nucleotide sequences comprising SEQ ID NO:1, SEQ ID NO:3, SEQ ID NO:5 and SEQ ID NO:7; and
b) nucleotide sequences comprising a nucleotide sequence having at least 95% homology to the nucleotide sequence set forth in SEQ ID NO:1, a nucleotide sequence having at least 95% homology to the nucleotide sequence set forth in SEQ ID NO:3, a nucleotide sequence having at least 95% homology to the nucleotide sequence set forth in SEQ ID NO:5 and a nucleotide sequence having at least 95% homology to the nucleotide sequence set forth in SEQ ID NO:7.

6. The nucleic acid molecule of claim 1 further comprising one or more nucleotide sequence encoding a cleavage site.

7. The nucleic acid molecule of claim 8 wherein the encoded cleavage site is rgrkrrs (SEQ ID NO:15), recognized by furin.

8. The nucleic acid molecule of claim 6 encoding an amino acid sequence selected from the group consisting of an amino acid sequence as set forth in SEQ ID NO:12, and an amino acid sequence having at least 95% homology to the amino acid sequence set forth in SEQ ID NO:12.

9. The nucleic acid molecule of claim 8 comprising a nucleotide sequence selected from the group consisting of SEQ ID NO:11, and a nucleotide sequence having at least 95% homology to the nucleotide sequence set forth in SEQ ID NO:11.

10. The nucleic acid molecule of claim 1 further comprising a nucleotide sequence encoding a viral 2A protein as set forth in SEQ ID NO:10.

11. The nucleic acid molecule of claim 1 further comprising a nucleotide sequence encoding an IgE leader sequence.

12. The nucleic acid molecule of claim 11 encoding an amino acid sequence selected from the group consisting of an amino acid sequence as set forth in SEQ ID NO:14, and an amino acid sequence having at least 95% homology to the amino acid sequence set forth in SEQ ID NO:14.

13. The nucleic acid molecule of claim 12 comprising a nucleotide sequence selected from the group consisting of SEQ ID NO:13, and a nucleotide sequence having at least 95% homology to the nucleotide sequence set forth in SEQ ID NO:13.

14. The nucleic acid molecule of claim 1, wherein the nucleic acid molecule is a plasmid.

15. An immunogenic composition comprising one or more plasmids of claim 14.

16. The immunogenic composition of claim 15 further comprising an adjuvant.

17. A method of generating an immune response against FMDV serotype O in an individual comprising administering to the individual an immunogenic composition of claim 15.

18. A method of preventing infection by FMDV serotype O in an individual comprising administering to the individual an immunogenic composition of claim 15.

19. A method of treating an individual who has been infected with FMDV serotype O comprising administering to the individual an immunogenic composition of claim 15.


SEQUENCE LISTING (16 sequences)

SEQ ID NO: 1 - DNA, 639 bp, artificial sequence
  nucleotide sequence encoding consensus FMDV-O VP1
accacctccaccggagagtccgccgacccagtgaccgccaccgtggagaactacggcggcgagacacaggtgcagcggcgccagcacaccgacgtgagcttcatcctggaccggttcgtgaaggtgaccccaaaggaccagatcaacgtgctggacctgatgcagatccctgcacacaccctggtgggcgccctgctgaggaccgccacctactacttcgccgacctggaggtggccgtgaagcacgagggcaacctgacctgggtgcctaacggagcaccagaggccgccctggacaacaccaccaacccaaccgcataccacaaggcccctctgaccaggctggccctgccatacaccgcccctcacagggtgctggcaaccgtgtacaacggcaactgcaagtacggagagtcccctgtgaccaacgtgcgcggcgacctgcaggtgctggcacagaaggcagcaaggaccctgcccaccagcttcaactacggcgccatcaaggccaccagggtgaccgagctgctgtacagaatgaagcgggccgagacatactgtcccagaccactgctggcaatccacccttccgaggcaaggcacaagcagaagatcgtggcaccagtgaagcagctgctg

SEQ ID NO: 2 - protein, 213 aa, artificial sequence
  amino acid sequence for consensus FMDV-O VP1
TTSTGESADPVTATVENYGGETQVQRRQHTDVSFILDRFVKVTPKDQINVLDLMQIPAHTLVGALLRTATYYFADLEVAVKHEGNLTWVPNGAPEAALDNTTNPTAYHKAPLTRLALPYTAPHRVLATVYNGNCKYGESPVTNVRGDLQVLAQKAARTLPTSFNYGAIKATRVTELLYRMKRAETYCPRPLLAIHPSEARHKQKIVAPVKQLL

SEQ ID NO: 3 - DNA, 654 bp, artificial sequence
  nucleotide sequence encoding consensus FMDV-O VP2
gacaagaagaccgaggagacaaccctgctggaggaccgcatcctgaccaccaggaacggccacaccaccagcaccacccagagctccgtgggagtgacctacggatacgcaaccgcagaggacttcgtgagcggaccaaacacctccggcctggagacaagggtggtgcaggcagagcggttcttcaagacccacctgttcgactgggtgacctccgaccctttcggcaggtgctacctgctggagctgcccaccgaccacaagggcgtgtacggcagcctgaccgactcctacgcctacatgagaaacggatgggacgtggaggtgaccgcagtgggaaaccagttcaacggcggatgcctgctggtggcaatggtgcccgagctgtgcagcatcgacaagagagagctgtaccagctgaccctgttccctcaccagttcatcaacccccggaccaacatgaccgcacacatcaccgtgcccttcgtgggagtgaaccgctacgaccagtacaaggtgcacaagccatggaccctggtggtcatggtggtggccccactgaccgtgaacaccgagggagccccacagatcaaggtgtacgccaacatcgcaccaaccaacgtgcacgtggcaggagagttccctagcaaggag

SEQ ID NO: 4 - protein, 218 aa, artificial sequence
  amino acid sequence for consensus FMDV-O VP2
DKKTEETTLLEDRILTTRNGHTTSTTQSSVGVTYGYATAEDFVSGPNTSGLETRVVQAERFFKTHLFDWVTSDPFGRCYLLELPTDHKGVYGSLTDSYAYMRNGWDVEVTAVGNQFNGGCLLVAMVPELCSIDKRELYQLTLFPHQFINPRTNMTAHITVPFVGVNRYDQYKVHKPWTLVVMVVAPLTVNTEGAPQIKVYANIAPTNVHVAGEFPSKE

SEQ ID NO: 5 - DNA, 660 bp, artificial sequence
  nucleotide sequence encoding consensus FMDV-O VP3
ggaatcttccctgtggcatgcagcgacggatacggcggcctggtgaccaccgaccccaagaccgccgacccagtgtacggcaaggtgttcaacccacccaggaacctgctgcccggcaggttcaccaacctgctggacgtggcagaggcatgtcccaccttcctgcacttcgacggcgacgtgccatacgtgaccaccaagaccgactccgacagggtgctggcacagttcgacctgtccctggcagccaagcacatgagcaacaccttcctggccggcctggcccagtactacacccagtacagcggcaccatcaacctgcacttcatgttcaccggcccaaccgacgccaaggccagatacatggtggcatacgcccctcccggaatggagccacctaagacccctgaggcagcagcacactgcatccacgccgagtgggacaccggcctgaacagcaagttcaccttctccatcccctacctgagcgccgcagactacgcatacaccgcatccgacgtggcagagacaaccaacgtgcagggctgggtgtgcctgttccagatcacccacggcaaggcagacggcgacgcactggtggtgctggccagcgccggcaaggacttcgacctgagactgccagtggacgcaaggacccag

SEQ ID NO: 6 - protein, 220 aa, artificial sequence
  amino acid sequence for consensus FMDV-O VP3
GIFPVACSDGYGGLVTTDPKTADPVYGKVFNPPRNLLPGRFTNLLDVAEACPTFLHFDGDVPYVTTKTDSDRVLAQFDLSLAAKHMSNTFLAGLAQYYTQYSGTINLHFMFTGPTDAKARYMVAYAPPGMEPPKTPEAAAHCIHAEWDTGLNSKFTFSIPYLSAADYAYTASDVAETTNVQGWVCLFQITHGKADGDALVVLASAGKDFDLRLPVDARTQ

SEQ ID NO: 7 - DNA, 219 bp, artificial sequence
  nucleotide sequence encoding consensus FMDV-O VP4
ggggcagggcagtcatctcccgcaacttggtcacagaaccagagcggcaacaccggctccatcatcaacaactactacatgcagcagtaccagaactccatggacacccagctgggcgacaacgccaccagcggcggctccaacgagggcagcaccgacaccacctccacccacaccaccaacacccagaacaacgactggttcagcaagctggcctcc

SEQ ID NO: 8 - protein, 73 aa, artificial sequence
  amino acid sequence for consensus FMDV-O VP4
GAGQSSPATWSQNQSGNTGSIINNYYMQQYQNSMDTQLGDNATSGGSNEGSTDTTSTHTTNTQNNDWFSKLAS

SEQ ID NO: 9 - DNA, 54 bp, artificial sequence
  nucleotide sequence encoding 2A
accctgaacttcgacctcctcaaactggctggcgatgtggaaagcaatcctgga

SEQ ID NO: 10 - protein, 18 aa, artificial sequence
  amino acid sequence for 2A
TLNFDLLKLAGDVESNPG

SEQ ID NO: 11 - DNA, 2238 bp, artificial sequence
  Nucleotide sequence encoding synthetic consensus FMDV-O comprising VP4, VP2, VP3, and VP1
atgggggcagggcagtcatctcccgcaacttggtcacagaaccagagcggcaacaccggctccatcatcaacaactactacatgcagcagtaccagaactccatggacacccagctgggcgacaacgccaccagcggcggctccaacgagggcagcaccgacaccacctccacccacaccaccaacacccagaacaacgactggttcagcaagctggcctccaggggcagaaagaggagatccgacaagaagaccgaggagacaaccctgctggaggaccgcatcctgaccaccaggaacggccacaccaccagcaccacccagagctccgtgggagtgacctacggatacgcaaccgcagaggacttcgtgagcggaccaaacacctccggcctggagacaagggtggtgcaggcagagcggttcttcaagacccacctgttcgactgggtgacctccgaccctttcggcaggtgctacctgctggagctgcccaccgaccacaagggcgtgtacggcagcctgaccgactcctacgcctacatgagaaacggatgggacgtggaggtgaccgcagtgggaaaccagttcaacggcggatgcctgctggtggcaatggtgcccgagctgtgcagcatcgacaagagagagctgtaccagctgaccctgttccctcaccagttcatcaacccccggaccaacatgaccgcacacatcaccgtgcccttcgtgggagtgaaccgctacgaccagtacaaggtgcacaagccatggaccctggtggtcatggtggtggccccactgaccgtgaacaccgagggagccccacagatcaaggtgtacgccaacatcgcaccaaccaacgtgcacgtggcaggagagttccctagcaaggagaggggaaggaagcggcgctccggaatcttccctgtggcatgcagcgacggatacggcggcctggtgaccaccgaccccaagaccgccgacccagtgtacggcaaggtgttcaacccacccaggaacctgctgcccggcaggttcaccaacctgctggacgtggcagaggcatgtcccaccttcctgcacttcgacggcgacgtgccatacgtgaccaccaagaccgactccgacagggtgctggcacagttcgacctgtccctggcagccaagcacatgagcaacaccttcctggccggcctggcccagtactacacccagtacagcggcaccatcaacctgcacttcatgttcaccggcccaaccgacgccaaggccagatacatggtggcatacgcccctcccggaatggagccacctaagacccctgaggcagcagcacactgcatccacgccgagtgggacaccggcctgaacagcaagttcaccttctccatcccctacctgagcgccgcagactacgcatacaccgcatccgacgtggcagagacaaccaacgtgcagggctgggtgtgcctgttccagatcacccacggcaaggcagacggcgacgcactggtggtgctggccagcgccggcaaggacttcgacctgagactgccagtggacgcaaggacccagaggggaagaaagaggagaagcaccacctccaccggagagtccgccgacccagtgaccgccaccgtggagaactacggcggcgagacacaggtgcagcggcgccagcacaccgacgtgagcttcatcctggaccggttcgtgaaggtgaccccaaaggaccagatcaacgtgctggacctgatgcagatccctgcacacaccctggtgggcgccctgctgaggaccgccacctactacttcgccgacctggaggtggccgtgaagcacgagggcaacctgacctgggtgcctaacggagcaccagaggccgccctggacaacaccaccaacccaaccgcataccacaaggcccctctgaccaggctggccctgccatacaccgcccctcacagggtgctggcaaccgtgtacaacggcaactgcaagtacggagagtcccctgtgaccaacgtgcgcggcgacctgcaggtgctggcacagaaggcagcaaggaccctgcccaccagcttcaactacggcgccatcaaggccaccagggtgaccgagctgctgtacagaatgaagcgggccgagacatactgtcccagaccactgctggcaatccacccttccgaggcaaggcacaagcagaagatcgtggcaccagtgaagcagctgctg

SEQ ID NO: 12 - protein, 746 aa, artificial sequence
  Amino acid sequence of synthetic consensus FMDV-O comprising VP4, VP2, VP3, and VP1
MGAGQSSPATWSQNQSGNTGSIINNYYMQQYQNSMDTQLGDNATSGGSNEGSTDTTSTHTTNTQNNDWFSKLASRGRKRRSDKKTEETTLLEDRILTTRNGHTTSTTQSSVGVTYGYATAEDFVSGPNTSGLETRVVQAERFFKTHLFDWVTSDPFGRCYLLELPTDHKGVYGSLTDSYAYMRNGWDVEVTAVGNQFNGGCLLVAMVPELCSIDKRELYQLTLFPHQFINPRTNMTAHITVPFVGVNRYDQYKVHKPWTLVVMVVAPLTVNTEGAPQIKVYANIAPTNVHVAGEFPSKERGRKRRSGIFPVACSDGYGGLVTTDPKTADPVYGKVFNPPRNLLPGRFTNLLDVAEACPTFLHFDGDVPYVTTKTDSDRVLAQFDLSLAAKHMSNTFLAGLAQYYTQYSGTINLHFMFTGPTDAKARYMVAYAPPGMEPPKTPEAAAHCIHAEWDTGLNSKFTFSIPYLSAADYAYTASDVAETTNVQGWVCLFQITHGKADGDALVVLASAGKDFDLRLPVDARTQRGRKRRSTTSTGESADPVTATVENYGGETQVQRRQHTDVSFILDRFVKVTPKDQINVLDLMQIPAHTLVGALLRTATYYFADLEVAVKHEGNLTWVPNGAPEAALDNTTNPTAYHKAPLTRLALPYTAPHRVLATVYNGNCKYGESPVTNVRGDLQVLAQKAARTLPTSFNYGAIKATRVTELLYRMKRAETYCPRPLLAIHPSEARHKQKIVAPVKQLL

SEQ ID NO: 13 - DNA, 2364 bp, artificial sequence
  Nucleotide sequence encoding synthetic consensus FMDV-O comprising VP4, VP2, VP3, and VP1 operably linked to nucleotide sequence encoding an IgE leader sequence
atggattggacatggattctgttcctcgtcgccgccgccactagggtgcattctggggcagggcagtcatctcccgcaacttggtcacagaaccagagcggcaacaccggctccatcatcaacaactactacatgcagcagtaccagaactccatggacacccagctgggcgacaacgccaccagcggcggctccaacgagggcagcaccgacaccacctccacccacaccaccaacacccagaacaacgactggttcagcaagctggcctccaggggcagaaagaggagatccgacaagaagaccgaggagacaaccctgctggaggaccgcatcctgaccaccaggaacggccacaccaccagcaccacccagagctccgtgggagtgacctacggatacgcaaccgcagaggacttcgtgagcggaccaaacacctccggcctggagacaagggtggtgcaggcagagcggttcttcaagacccacctgttcgactgggtgacctccgaccctttcggcaggtgctacctgctggagctgcccaccgaccacaagggcgtgtacggcagcctgaccgactcctacgcctacatgagaaacggatgggacgtggaggtgaccgcagtgggaaaccagttcaacggcggatgcctgctggtggcaatggtgcccgagctgtgcagcatcgacaagagagagctgtaccagctgaccctgttccctcaccagttcatcaacccccggaccaacatgaccgcacacatcaccgtgcccttcgtgggagtgaaccgctacgaccagtacaaggtgcacaagccatggaccctggtggtcatggtggtggccccactgaccgtgaacaccgagggagccccacagatcaaggtgtacgccaacatcgcaccaaccaacgtgcacgtggcaggagagttccctagcaaggagaggggaaggaagcggcgctccggaatcttccctgtggcatgcagcgacggatacggcggcctggtgaccaccgaccccaagaccgccgacccagtgtacggcaaggtgttcaacccacccaggaacctgctgcccggcaggttcaccaacctgctggacgtggcagaggcatgtcccaccttcctgcacttcgacggcgacgtgccatacgtgaccaccaagaccgactccgacagggtgctggcacagttcgacctgtccctggcagccaagcacatgagcaacaccttcctggccggcctggcccagtactacacccagtacagcggcaccatcaacctgcacttcatgttcaccggcccaaccgacgccaaggccagatacatggtggcatacgcccctcccggaatggagccacctaagacccctgaggcagcagcacactgcatccacgccgagtgggacaccggcctgaacagcaagttcaccttctccatcccctacctgagcgccgcagactacgcatacaccgcatccgacgtggcagagacaaccaacgtgcagggctgggtgtgcctgttccagatcacccacggcaaggcagacggcgacgcactggtggtgctggccagcgccggcaaggacttcgacctgagactgccagtggacgcaaggacccagaggggaagaaagaggagaagcaccacctccaccggagagtccgccgacccagtgaccgccaccgtggagaactacggcggcgagacacaggtgcagcggcgccagcacaccgacgtgagcttcatcctggaccggttcgtgaaggtgaccccaaaggaccagatcaacgtgctggacctgatgcagatccctgcacacaccctggtgggcgccctgctgaggaccgccacctactacttcgccgacctggaggtggccgtgaagcacgagggcaacctgacctgggtgcctaacggagcaccagaggccgccctggacaacaccaccaacccaaccgcataccacaaggcccctctgaccaggctggccctgccatacaccgcccctcacagggtgctggcaaccgtgtacaacggcaactgcaagtacggagagtcccctgtgaccaacgtgcgcggcgacctgcaggtgctggcacagaaggcagcaaggaccctgcccaccagcttcaactacggcgccatcaaggccaccagggtgaccgagctgctgtacagaatgaagcgggccgagacatactgtcccagaccactgctggcaatccacccttccgaggcaaggcacaagcagaagatcgtggcaccagtgaagcagctgctgaggggacgcaagaggcgctcaaccctgaacttcgacctcctcaaactggctggcgatgtggaaagcaatcctgga

SEQ ID NO: 14 - protein, 788 aa, artificial sequence
  Amino acid sequence of a synthetic consensus FMDV-O comprising VP4, VP2, VP3, and VP1 operably linked to an IgE leader sequence
MDWTWILFLVAAATRVHSGAGQSSPATWSQNQSGNTGSIINNYYMQQYQNSMDTQLGDNATSGGSNEGSTDTTSTHTTNTQNNDWFSKLASRGRKRRSDKKTEETTLLEDRILTTRNGHTTSTTQSSVGVTYGYATAEDFVSGPNTSGLETRVVQAERFFKTHLFDWVTSDPFGRCYLLELPTDHKGVYGSLTDSYAYMRNGWDVEVTAVGNQFNGGCLLVAMVPELCSIDKRELYQLTLFPHQFINPRTNMTAHITVPFVGVNRYDQYKVHKPWTLVVMVVAPLTVNTEGAPQIKVYANIAPTNVHVAGEFPSKERGRKRRSGIFPVACSDGYGGLVTTDPKTADPVYGKVFNPPRNLLPGRFTNLLDVAEACPTFLHFDGDVPYVTTKTDSDRVLAQFDLSLAAKHMSNTFLAGLAQYYTQYSGTINLHFMFTGPTDAKARYMVAYAPPGMEPPKTPEAAAHCIHAEWDTGLNSKFTFSIPYLSAADYAYTASDVAETTNVQGWVCLFQITHGKADGDALVVLASAGKDFDLRLPVDARTQRGRKRRSTTSTGESADPVTATVENYGGETQVQRRQHTDVSFILDRFVKVTPKDQINVLDLMQIPAHTLVGALLRTATYYFADLEVAVKHEGNLTWVPNGAPEAALDNTTNPTAYHKAPLTRLALPYTAPHRVLATVYNGNCKYGESPVTNVRGDLQVLAQKAARTLPTSFNYGAIKATRVTELLYRMKRAETYCPRPLLAIHPSEARHKQKIVAPVKQLLRGRKRRSTLNFDLLKLAGDVESNPG

SEQ ID NO: 15 - protein, 7 aa, artificial sequence
  furin cleavage site
RGRKRRS

SEQ ID NO: 16 - protein, 18 aa, artificial sequence
  IgE leader sequence
MDWTWILFLVAAATRVHS